(19) United States
(12) Patent Application Publication
(10) Pub. No.: US 2026/0092930 A1
(43) Pub. Date: Apr. 2, 2026

(54) DESMIN TYROSINE PHOSPHORYLATED MUTANT AND PANCREATIC CANCER

(71) Applicants: KENDRICK LABORATORIES, INC., MADISON, WI (US); CLARKSON UNIVERSITY, Potsdam, NY (US)

(72) Inventors: Nancy KENDRICK, Monona, WI (US); Jon JOHANSEN, Madison, WI (US); Matt HOELTER, Oregon, WI (US); Andrew KOLL, Madison, WI (US); Costel C. DARIE, Madison, WI (US)

(21) Appl. No.: 19/348,598

(22) Filed: Oct. 2, 2025

Related U.S. Application Data
(60) Provisional application No. 63/702,495, filed on Oct. 2, 2024.

(51) Int. Cl. G01N 33/68; A61K 38/00; C07K 14/47; G01N 1/28

(52) U.S. Cl. CPC G01N 33/6893; C07K 14/4716; G01N 1/28; A61K 38/00; G01N 2800/06; G01N 2800/32

(57) ABSTRACT

The present invention provides methods for detecting a mutant desmin protein comprising a phosphorylated D399Y mutation. In some embodiments, the methods are used to diagnose cancer in a subject. Also provided are compositions comprising or constructs encoding a fragment of the mutant desmin protein with a phosphorylated tyrosine residue at position 399 corresponding to a D399Y mutation and methods of using these compositions to treat cancer.

### CROSS-REFERENCE TO RELATED APPLICATIONS

This application claims priority to U.S. Provisional Application No. 63/702,495, filed Oct. 2, 2024, which is hereby incorporated by reference in its entirety.

### REFERENCE TO SEQUENCE LISTING

The contents of the electronic sequence listing (16997500006.xml; Size: 7,744 bytes; and Date of Creation: Sep. 26, 2025) is herein incorporated by reference in its entirety.

### FILED OF THE INVENTION

The present disclosure relates to cancer biomarkers and therapeutic compositions, and more particularly to methods for detecting a mutant desmin protein comprising a phosphorylated D399Y mutation as a biomarker for pancreatic ductal adenocarcinoma and therapeutic compositions comprising fragments of the mutant desmin protein for treating cancer.

### BACKGROUND

Pancreatic ductal adenocarcinoma (PDAC) is the most common type of pancreatic cancer, accounting for more than 80% of cases. This cancer develops in cells that line that ducts that carry secretions (e.g., digestive enzymes, bicarbonate) away from the pancreas. PDAC is one of the most aggressive and deadliest forms of cancer, with a 5-year survival rate of only 12% [1, 2]. It often doesn't cause symptoms until it has spread to other organs, so it's rarely found in its early stages when it's most treatable. While considerable knowledge has been gained from genomic [18], transcriptomic [19] and proteogenomic [20] analysis of PDAC tumors, actionable biomarkers for this deadly cancer remain elusive [21]. Accordingly, there is a remaining need in the art for PDAC biomarkers that enable earlier detection of this cancer.

### SUMMARY

In a first aspect, the present invention provides methods for detecting a mutant desmin protein comprising a phosphorylated D399Y mutation. The methods comprise: (a) obtaining a sample from a subject, and (b) detecting the mutant desmin protein in the sample by detection of the tyrosine residue at position 399 of desmin that is phosphorylated. The sample may be a pancreatic tissue sample and the presence of the mutation is associated with pancreatic cancer including pancreatic ductal adenocarcinoma (PDAC). The method can be applied, in addition to PDAC, to any other cancer. The method and this mutation is also associated with myopathies, including cardiomyopathies.

In a second aspect, the present invention provides compositions comprising a fragment of the mutant desmin protein of SEQ ID NO: 6 that comprises amino acid residue Y399. The tyrosine at position 399 of desmin may be phosphorylated. The fragment may comprise the amino acid sequence MALYVEIATYR (SEQ ID NO: 3). The composition may further comprise a pharmaceutically acceptable carrier. The composition may be formulated for therapeutic use in treating PDAC.

In a third aspect, the present invention provides polynucleotides encoding a mutant desmin protein fragment disclosed herein. The polynucleotides may be in a construct or gene therapy vector and may include a promoter operably connected to the polynucleotide encoding the mutant desmin protein or portion thereof.

In a fourth aspect, the present invention provides methods of treating a cancer or a myopathy such as a cardiomyopathy in a subject. The methods comprise administering a composition or polynucleotide described herein to the subject. The method comprises administering to the subject a composition comprising a fragment of a mutant desmin protein, wherein the fragment comprises a tyrosine residue at position 399 corresponding to a D399Y mutation in wild-type desmin protein. The cancer may be PDAC or any other cancer. The fragment may comprise the amino acid sequence MALYVEIATYR. The composition may be administered intravenously. The subject may have a cancer expressing a mutant desmin protein comprising a phosphorylated D399Y mutation.

### BRIEF DESCRIPTION OF THE DRAWINGS

FIG. 1 shows western blots for KRAS, YAP, Src, and phosphotyrosine (pTyr) in six pancreatic tumor (“T”; P1-P5 & P0) and five normal adjacent (“N”; P1-P5) tissue samples. Equal protein loading (40 μg/lane) was used for normalization [12]. The heavily loaded molecular weight markers are myosin, 220 kDa; phosphorylase A, 94 kDa; catalase, 60 kDa; actin, 43 kDa; carbonic anhydrase, 29 kDa; and lysozyme, 14 kDa. Red arrow highlights an unknown pTyr-protein of interest.

FIG. 2 shows two-dimensional (2D) western blots for pTyr in the pancreatic tumor samples P1-T and P2-T and the normal adjacent tissue (NAT) samples P1-N and P2-N. Red arrows mark a ˜60 kDa pTyr-protein in the P1-T and P2-T samples that was observed after a 15-minute film exposure.

FIG. 3 shows a Coomassie-stained 2D gel analysis of the pancreatic tumor samples P1-T (A) and P2-T (C) and the NAT samples P1-N(B) and P2-N(D). Red arrows mark a ˜60 kDa pTyr protein with four charge-shift isoforms that were substantially increased in the P1-T and P2-T tumor samples as compared to in the NAT control samples.

FIG. 4 shows blown-up images of the area of interest in the 2D gels. The pTyr western blot is shown on the left, the Coomassie blue-stained gel is on the right, and a Coomassie/western blot overlay is in the center. The latter was used to cut spots from the Coomassie gel for identification by mass spectrometry (MS).

FIG. 5 shows MS and MS/MS spectra that correspond to the peptide mALDVEIATYRK (SEQ ID NO: 2). (A) Extracted ion chromatogram (XIC) for the precursor ion with m/z of 475.93(3+) that corresponds to peptides mALpYVEIATYR (SEQ ID NO: 4) and mALDVEIATYRK (SEQ ID NO: 2). (B) Total ion chromatogram (TIC) from which the XIC was obtained. C: (C) MS spectrum in which both (3+) and (2+) precursor ions that correspond to peptides mALpYVEIATYR (SEQ ID NO: 4) and mALDVEIATYRK (SEQ ID NO: 2) were identified with m/z of 475.93 (3+) and 713.41 (2+).

FIG. 6 shows an MS/MS spectrum that shows the fragmentation of the precursor ion with m/z 713.40 (2+) that corresponds to peptides mALpYVEIATYR (SEQ ID NO: 4) and mALDVEIATYRK (SEQ ID NO: 2). The fragment ions that correspond to the peptide mALpYVEIATYR (SEQ ID NO: 4) are marked with blue rectangles in the top spectrum. Fragment ions that are unique to this peptide are marked with a red star. The fragment ions that correspond to the peptide mALDVEIATYRK (SEQ ID NO: 2) are marked with red rectangles in the bottom spectrum. Fragment ions that are unique to this peptide are marked with a blue star.

FIGS. 7A-7C show Mascot database searches that support the identification of both the wild-type desmin protein and the novel mutant desmin protein. (7A) MS/MS fragmentation of the peptide mALDVEIATYRK (SEQ ID NO: 2), identified through a precursor ion with m/z 713.39 (2+). The y and b ions identified from the Mascot database search are highlighted in red. (7B) MS/MS fragmentation of the peptide mALpYVEIATYR (SEQ ID NO: 4), identified through a precursor ion with m/z 713.41 (2+). The y and b ions identified from the Mascot database search are highlighted in red. (7C) Amino acid sequences of wild-type (top; SEQ ID NO: 1) and mutant (bottom; SEQ ID NO: 6) desmin proteins. Note: While the main precursor ion was detected in the MS scan as 713.39 (2+), multiple precursor ions with m/z of 713.39 (2+), 713.40 (2+), and 713.41 (2+) were selected for MS/MS. They produced a series of b and y ions that were shared by the peptides mALDVEIATYRK (SEQ ID NO: 2) and mALpYVEIATYR (SEQ ID NO: 4), as well as a series of ions that were unique to either mALDVEIATYRK (SEQ ID NO: 2) or mALpYVEIATYR (SEQ ID NO: 4).

FIGS. 8A, 8B, 8C, 8D, and 8E show expanded MS/MS spectra from FIG. 6.

FIGS. 9A, 9B, 9C, 9D, and 9E show expanded MS/MS spectra of the precursor ion with m/z of 475.93 (3+) (top), which corresponds to the peptide mALpYVEIATYR (SEQ ID NO: 4), and the precursor ion with m/z of 713.41 (2+), which that correspond to the peptide mALDVEIATYRK (SEQ ID NO: 2). Note: The precursor ion with m/z of 475.93 (3+) and its MS/MS further confirm the identity of the peptides mALDVEIATYRK (SEQ ID NO: 2) and mALpYVEIATYR (SEQ ID NO: 4), detected through precursor ion with m/z of 713.41 (2+), and shown in FIGS. 8A-8E.

### DETAILED DESCRIPTION

The present invention provides methods for detecting a mutant desmin protein comprising a phosphorylated D399Y mutation. In some embodiments, the methods are used to diagnose cancer in a subject. Also provided are compositions comprising or polynucleotides encoding a fragment of the mutant desmin protein and methods of using these compositions to treat cancer. These methods and compositions may be particularly applicable to pancreatic ductal adenocarcinoma (PDAC), a highly aggressive form of cancer with limited treatment options and poor patient outcomes. The mutant desmin protein represents a novel biomarker that may facilitate earlier detection and targeted therapeutic intervention in cancer patients

As is described in the Examples, the present inventors have identified a novel mutant protein that is expressed in pancreatic ductal adenocarcinoma (PDAC) tumors. The inventors identified this protein while performing a protein-based screen for oncogenic drivers of PDAC. Specifically, they used a combination of 1D/2D phosphotyrosine (pTyr) western blotting and nano liquid chromatography tandem mass spectrometry (NanoLC-MS/MS) to analyze homogenates of PDAC tumor, looking for aberrant expression of receptor tyrosine kinases and proteins that they regulate. In the process, they discovered a novel, abundant 55 kDa protein in 2/6 tumor samples and identified it as mutant form of the intermediate filament protein desmin. A proteomic analysis of the mutant desmin protein revealed that it contains the mutation D399Y and that this mutated tyrosine was phosphorylated in the PDAC samples. The inventors suggest that this mutant desmin protein may be a useful biomarker for the diagnosis of PDAC. Further, this mutant desmin protein may play a role in promoting PDAC metastasis, and they suggest that cancers that express it could potentially be treated using a decoy peptide that reduces its phosphorylation.

### Methods for Detecting Mutant Desmin:

In a first aspect, the present invention provides methods for detecting a mutant desmin protein comprising a phosphorylated D399Y mutation. The methods comprise: (a) obtaining a sample from a subject, and (b) detecting the mutant desmin protein in the sample.

The terms “protein,” “polypeptide,” and “peptide” are used interchangeably herein to refer to a polymer of amino acid residues connected by peptide bonds between the alpha-amino and carboxy groups of adjacent residues.

Desmin is a type III intermediate filament protein that is expressed in muscle (i.e., cardiac, skeletal, and smooth) and endothelial cells in vertebrates. Intermediate filaments are cytoskeletal proteins that provide structure and mechanical strength to cells. They are also essential for anchoring cells to other cells and to the extracellular matrix. The protein functions by connecting myofibrils to each other and to the plasma membrane, thereby maintaining proper cellular architecture during muscle contraction and relaxation. Desmin also serves as a marker for activated pancreatic stellate cells, which are non-cancerous mesenchymal cells present in tumor stroma that contribute to the formation of dense fibrotic tissue in pancreatic cancer. The full-length sequence of the wild-type, human desmin protein is provided as SEQ ID NO: 1. The term “wild-type” is used herein to refer to the form of a protein that most commonly occurs in nature.

In the methods of the present invention, a mutant form of desmin is detected. The term “mutant” is used herein to refer to a form of a protein that comprises a mutation. The term “mutation” refers to a difference in amino acid sequence relative to a reference sequence, such as the sequence of the corresponding wild-type protein. Mutations include insertions, deletions, and substitutions of amino acid residues relative to a reference sequence.

As is described in the Examples, the inventors identified a mutant desmin protein that comprises a D399Y mutation, meaning that the aspartic acid found at residue 399 in the wild-type protein has been substituted with a tyrosine in this mutant protein. The full-length sequence of this mutant desmin protein is provided as SEQ ID NO: 6. In some embodiments, the mutant desmin protein has at least 50%, 55%, 60%, 65%, 70%, 75%, 80%, 85%, 90%, 95%, 98%, or 99% sequence identity to SEQ ID NO: 6. In some embodiments, the mutant desmin protein comprises only the single D399Y mutation relative to the wild-type desmin sequence of SEQ ID NO: 1. In these embodiments, the mutant desmin protein may comprise, consist, or consist essentially of SEQ ID NO: 6. The term “mutant desmin protein” is used herein to mean a form of desmin protein that comprises a mutation relative to wild-type desmin protein. Any mutant desmin protein described herein comprises at least a D399Y mutation, wherein the aspartic acid residue found at position 399 in wild-type desmin is substituted with a tyrosine residue. The mutant desmin protein may be phosphorylated at the tyrosine residue at position 399, creating a phosphotyrosine modification that distinguishes the mutant protein from wild-type desmin and provides a molecular signature for detection and therapeutic targeting. The presence of this phosphorylated mutant desmin protein in cancer tissues may serve as a distinctive molecular signature that distinguishes malignant cells from normal adjacent tissue.

Without being bound by theory, pancreatic ductal adenocarcinoma is characterized by extensive desmoplasia, a process involving the formation of dense fibrotic tissue that comprises a substantial portion of the tumor mass. This desmoplastic reaction involves complex interactions between cancer cells and stromal components, including pancreatic stellate cells that express desmin. The identification of mutant phosphorylated desmin in pancreatic cancer tissues suggests a potential role for this protein in the pathological processes underlying tumor development and progression. The mutant desmin protein may contribute to epithelial-mesenchymal transition, a cellular process that enables cancer cells to acquire migratory and invasive properties associated with metastasis.

Current diagnostic and therapeutic approaches for pancreatic cancer face significant challenges due to the heterogeneous nature of the disease and the lack of reliable biomarkers for early detection. The mutant desmin protein comprising a phosphorylated D399Y mutation may address these limitations by providing a specific molecular target for both diagnostic and therapeutic applications. Detection methods utilizing this biomarker may enable identification of cancer patients who would benefit from targeted interventions, while therapeutic compositions based on fragments of the mutant protein may offer new treatment strategies for managing pancreatic cancer and potentially other malignancies expressing this aberrant protein

“Percentage of sequence identity” is determined by comparing two optimally aligned sequences over a comparison window. The aligned sequences may comprise additions or deletions (i.e., gaps) relative to each other for optimal alignment. The percentage is calculated by determining the number of matched positions at which an identical nucleic acid base or amino acid residue occurs in both sequences, dividing the number of matched positions by the total number of positions in the window of comparison and multiplying the result by 100. Protein and nucleic acid sequence identities can be evaluated using the Basic Local Alignment Search Tool (“BLAST”), which is well known in the art (Karlin and Altschul, Methods for assessing the statistical significance of molecular sequence features by using general scoring schemes. Proc. Natl. Acad. Sci. USA (1990) 87: 2267-2268; Altschul et al. Gapped BLAST and PSI-BLAST: a new generation of protein database search programs. Nucl. Acids Res. (1997) 25: 3389-3402). BLAST identifies homologous sequences by identifying similar segments between a query amino acid or nucleic acid sequence and a test sequence, which is preferably obtained from a protein or nucleic acid sequence database. BLAST can be used with the default parameters or with modified parameters provided by the user.

The mutant desmin protein that the inventors identified is phosphorylated on the mutated Y399 residue. “Phosphorylation” is the attachment of a phosphate group to a molecule. Protein phosphorylation is the major mechanism through which protein activity, stability, interactions, and sub-cellular localization is regulated. It can occur on serine, threonine, and tyrosine side chains through phosphoester bond formation, on histidine, lysine, and arginine through phosphoramidate bond formation, and on aspartic acid and glutamic acid through mixed anhydride linkages.

In step (a) of the present methods, a sample is obtained from a subject. A “sample” is a small amount of material that is taken from a subject for use in an analysis. A sample may comprise tissue, fluid, cells, or other materials. Examples of suitable tissues include, but are not limited to, organs, skin, muscle, and bone. Examples of suitable fluids include, but are not limited to, saliva, blood, serum, plasma, urine, stool, and cerebrospinal fluid. Sample acquisition may encompass various tissue types, with pancreatic tissue samples representing a particularly relevant source for detecting the mutant desmin protein in the context of pancreatic ductal adenocarcinoma. In some cases, samples may be obtained through biopsy procedures, surgical resection, or autopsy collection, depending on the clinical circumstances and diagnostic requirements. The samples may include tumor tissue, normal adjacent tissue, or both types of specimens to enable comparative analysis of protein expression patterns. Sample collection procedures may follow standard medical protocols to ensure tissue integrity and preserve protein structure for subsequent analytical procedures.

The inventors identified the mutant desmin protein in tumor samples. Thus, in some embodiments, the sample is a cancer biopsy. A “cancer biopsy” is a sample that is collected to determine whether or not it is cancerous. Cancer biopsies include both solid tumor samples and liquid biopsies (i.e., blood samples collected to be analyzed for circulating tumor DNA). Solid tumor samples include samples collected via a needle or incisional biopsy and portions of surgically resected tumors. Cancer biopsies may comprise fresh, frozen, or formalin fixed paraffin embedded (FFPE) material. In addition to cancer, the inventors postulate that the mutation, specifically phosphorylated mutated D399Y also applies to myopathies, including cardiomyopathies.

The “subject” to which the methods are applied may be any vertebrate animal. Suitable subjects include, but are not limited to, humans, cows, horses, sheep, pigs, goats, rabbits, dogs, cats, bats, mice, and rats. In certain embodiments, the methods may be performed on lab animals (e.g., mice and rats) for research purposes. In other embodiments, the methods are used to treat commercially important farm animals (e.g., cows, horses, pigs, rabbits, goats, sheep, and chickens) or companion animals (e.g., cats and dogs). In preferred embodiments, the subject is a human. In some embodiments, the subject is suspected of having cancer.

In some embodiments, the sample is processed prior to step (b). For example, the sample may be homogenized in buffer that facilitates complete protein solubilization while preventing degradation of phosphorylated residues. In some cases, the homogenization process may be performed using motorized glass-teflon homogenizers or similar mechanical disruption devices to achieve thorough tissue breakdown. The homogenization procedure may be conducted at low temperatures, such as on ice with ice-cold reagents, to minimize protein degradation and preserve post-translational modifications during the extraction process. The buffer used may include sodium dodecyl sulfate (SDS) as a primary component. In some cases, the homogenization buffer may comprise osmotic lysis buffer containing 10 mM Tris and 0.3% SDS, supplemented with various protective agents to maintain protein integrity. The buffer system may incorporate protease inhibitors including AEBSF, leupeptin, E-64, 5 mM EDTA, and benzamidine to prevent proteolytic degradation of the target proteins. Additionally, the buffer may contain phosphatase inhibitors I and II to preserve phosphorylation states of tyrosine residues, along with RNase and DNase to remove nucleic acids that might interfere with protein analysis. The buffer composition may further include 5 mM MgCl₂ to support enzymatic activities of the nucleases during the homogenization process.

Following initial homogenization, the sample may undergo dilution with additional SDS buffer to achieve optimal protein concentrations for analysis. The dilution process may involve mixing the homogenized tissue with SDS buffer containing 5% SDS, 10% glycerol, and 62.5 mM Tris at pH 6.8 in a 1:1 ratio. This dilution step may serve to standardize protein concentrations across different samples while maintaining the denaturing conditions necessary for complete protein extraction. The glycerol component may provide cryoprotective properties and help stabilize protein structures during storage, while the Tris buffer system may maintain appropriate pH conditions for protein stability and subsequent analytical procedures.

The sample may be boiled or subjected to a thermal treatment prior to the detection step as well. This step may be useful to enhance protein solubilization and ensure complete extraction of membrane-associated and cytoskeletal proteins. The homogenized and diluted samples may be subjected to boiling in a water bath for five minutes to maximize protein solubilization and disrupt protein-protein interactions that might otherwise limit extraction efficiency. This boiling step may be particularly beneficial for extracting intermediate filament proteins such as desmin, which may form stable cytoskeletal networks that resist conventional extraction methods. The elevated temperature treatment may cause protein denaturation and facilitate the release of proteins from cellular structures, thereby improving the overall yield of extractable protein for subsequent analysis. Following the boiling treatment, samples may be cooled and processed for protein concentration determination using standard biochemical assays before proceeding to detection methods for the mutant desmin protein. The term “homogenization” refers to a process in which a material is broken down into smaller, similarly sized particles. Homogenization of a tissue sample serves to break down the architecture of the tissue and lyse cells to release intracellular analytes, such as proteins. Tissue homogenization may be accomplished using mechanical (e.g., mortar and pestle, blender, rotor-stator), high-pressure (e.g., French press), chemical (e.g., detergent), ultrasonic, freeze-thaw, or laser-based methods.

A “buffer” is a solution that resists changes in pH when acid or alkali is added to it. Preferably, the buffer used in the present methods comprises sodium dodecyl sulfate (SDS), which is a water-soluble detergent that helps disperse hydrophobic proteins into an aqueous solution. The buffer may further comprise additional reagents that help to stabilize proteins, such as protease inhibitors and phosphatase inhibitors. Examples of suitable protease inhibitors include, without limitation, aprotinin, antipain, bestatin, benzamidine, E-64, EDTA, leupeptin, pepstatin A, 4-(2-Aminoethyl)-benzenesulfonylfluoride hydrochloride (AEBSF), and phenylmethylsulfonyl fluoride (PMSF). Examples of suitable phosphatase inhibitors include, without limitation, sodium fluoride, sodium orthovanadate, beta-glycerophosphate, sodium pyrophosphate.

“Boiling” is the process of bringing a liquid to a temperature at which it bubbles and turns to vapor. A sample can be boiled by heating it. In the Examples, the inventors boiled their samples by placing them in a water bath for five minutes. Thus, in some embodiments the sample is boiled for five minutes.

In step (b) of the methods, the mutant desmin protein is detected in the sample. The amino acid substitution creates a novel phosphorylation site that may be targeted by cellular kinases, resulting in the formation of a phosphotyrosine residue that alters the biochemical properties of the protein. The phosphorylated tyrosine residue at position 399 may influence the overall charge distribution of the mutant desmin protein, affecting its migration patterns during electrophoretic separation and enabling differentiation from the wild-type protein through various analytical techniques. The mutant desmin protein may be detected using mass spectroscopy, 2D gel electrophoresis, or using an antibody-based detection method, such as western blotting, immunohistochemistry, or enzyme-linked immunosorbent assay (ELISA).

When analyzed using two-dimensional gel electrophoresis, the mutant desmin protein appears as a cluster of four isoforms due to post-translational modifications that change the isoelectric point of the protein. These four isoforms may result from various chemical modifications including cysteine oxidation, carbamylation, glycosylation, or differential phosphorylation states that occur during cellular processing of the mutant protein. The charge isoform clusters represent a common phenomenon observed in proteomic analysis, where a single protein species may migrate to multiple positions on a two-dimensional gel based on subtle differences in charge characteristics. The formation of multiple isoforms may provide additional specificity for detecting the mutant desmin protein, as the characteristic four-spot pattern may serve as a distinctive signature that distinguishes the mutant protein from other cellular proteins with similar molecular weight.

The molecular weight characteristics of the mutant desmin protein may remain similar to wild-type desmin, with the protein migrating at approximately 55 kDa during electrophoretic analysis. However, the phosphorylation of the tyrosine residue at position 399 may introduce additional mass that may be detectable through high-resolution mass spectrometry techniques. The phosphate group addition may contribute approximately 80 Daltons to the molecular weight of the protein, creating a measurable difference that may be utilized for specific identification of the phosphorylated mutant form. The combination of the characteristic molecular weight, isoelectric point variations, and specific amino acid sequence features may provide multiple parameters for reliable detection and confirmation of the mutant desmin protein comprising a phosphorylated D399Y mutation in biological samples.

In preferred embodiments, the mutant desmin protein is detected using an antibody that specifically binds to it and identify the phosphorylated tyrosine at position 399 or the surrounding epitope in the mutant form of desmin. The term “antibody” refers to a protein that comprises at least one antigen-binding domain from an immunoglobulin molecule. Suitable antibodies include, without limitation, whole antibodies (e.g., IgG, IgA, IgE, IgM, IgD), monoclonal antibodies, polyclonal antibodies, chimeric antibodies, humanized antibodies, and antibody fragments, including single chain variable fragments (scFv), single domain antibodies, and antigen-binding fragments. The antibody used with the present invention may specifically bind to the mutant desmin protein via recognition of the phosphorylated D399Y mutation.

As used herein, the term “specific” refers to the ability of a protein to bind one molecule in preference to other molecules. Under appropriate conditions, a protein that specifically binds to a target molecule will bind to that target molecule without binding to other molecules present in a sample in a significant amount. Specific binding can mean binding to a target molecule with an affinity that is at least 25% greater, at least 50% greater, at least 100% (2-fold) greater, at least ten times greater, at least 20-times greater, or at least 100-times greater than the affinity to other molecules.

The inventors identified the mutant desmin protein as a cancer-specific protein, but similar mutations may be associated with myopathies such as cardiomyopathies. Thus, this protein may be a biomarker of cancer and in particular may be used in methods of diagnosing pancreatic ductal adenocarcinoma. Accordingly, in some embodiments, the methods are performed to diagnose cancer in a subject. In these embodiments, the subject is diagnosed with cancer if the mutant desmin protein is detected in the sample from the subject. Diagnosing pancreatic ductal adenocarcinoma in subjects through identification of the mutant desmin protein comprising a phosphorylated D399Y mutation in biological samples is thus also provided here. The diagnostic application may rely on the distinctive presence of the mutant desmin protein in tumor tissues compared to normal adjacent tissues, providing a molecular signature that distinguishes malignant cells from healthy pancreatic tissue. The diagnostic process may involve obtaining tissue samples from subjects suspected of having pancreatic cancer, followed by analysis using the detection methods to determine the presence or absence of the mutant desmin protein. The diagnostic determination may be based on the detection of phosphorylated tyrosine residues at position 399 of the desmin protein, which may serve as a specific biomarker for pancreatic ductal adenocarcinoma. The diagnostic approach may complement existing clinical assessment methods by providing molecular-level confirmation of cancer presence and potentially enabling earlier detection of malignant transformation in pancreatic tissues

Intermediate filaments (IFs) reorganize in response to differentiation related signals [38], and several IFs have been implicated in cancer epithelial mesenchymal transformation (EMT) and metastasis [39]. Further, serine/threonine phosphorylation of the IFs desmin and vimentin has been shown to cause disassembly and/or softening of the cell cytoskeleton, which could promote EMT and cell migration [41]. Thus, the inventors hypothesize that the phosphorylated mutant desmin protein may play a role in promoting tumor metastasis. Accordingly, in some embodiments, the subject is diagnosed with metastatic cancer if the mutant desmin protein is detected. “Metastatic cancer” is cancer that has spread from its original location to another part of the body. The ability to diagnose metastatic cancer or lack thereof or the risk of metastasis may ais in developing or recommending treatment options for a subject diagnosed with the cancer. The molecular characterization provided by mutant desmin protein detection may enable more precise staging of cancer patients and inform treatment strategies that address both local and systemic disease components. The diagnostic approach may also facilitate monitoring of treatment response by assessing changes in mutant desmin protein levels following therapeutic interventions.

The diagnostic applications may extend to monitoring disease progression and treatment response through serial analysis of mutant desmin protein levels in accessible tissue samples or liquid biopsy specimens. The longitudinal monitoring approach may enable assessment of changes in mutant desmin protein expression following therapeutic interventions, providing molecular markers of treatment efficacy that complement conventional imaging and clinical assessment methods. The diagnostic method may be adapted for analysis of circulating tumor cells or extracellular vesicles that may contain the mutant desmin protein, potentially enabling non-invasive monitoring of disease status through blood-based assays. The diagnostic utility may include identification of patients who may benefit from targeted therapies directed against the cellular pathways involving the mutant desmin protein, supporting personalized treatment approaches based on molecular tumor characteristics. The diagnostic method may contribute to clinical trial stratification by identifying patient populations with specific molecular features that may predict response to experimental therapeutic agents targeting desmin-related pathways or epithelial mesenchymal transformation processes.

As used herein, the term “cancer” refers to diseases in which abnormal cells divide uncontrollably. The methods of the present invention may be used to diagnose any type of cancer that expresses the mutant desmin protein. Examples of suitable cancer types include, without limitation, breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma and peripheral neuroepithelioma.

The inventors identified the mutant desmin protein described herein in pancreatic ductal adenocarcinoma (PDAC) tumor samples. Accordingly, in some embodiments, the sample comprises pancreatic tissue and the subject is diagnosed with PDAC if the mutant desmin protein is detected. Pancreatic ductal adenocarcinoma (PDAC) is a highly aggressive and lethal cancer that originates in the ducts that carry secretions (e.g., enzymes, bicarbonate) away from the pancreas.

In embodiments in which the methods comprise diagnosing a subject with cancer, the methods may further comprise: (c) treating the subject with a cancer treatment. As used herein, “treating” describes something that is done to a subject to combat a disease. Treating includes administering a treatment to prevent the onset of the symptoms or complications, to alleviate symptoms or complications, or to eliminate the disease. For example, treating a cancer in a subject includes reducing, repressing, delaying, or preventing the growth of the cancer, reducing tumor volume or number, and/or preventing, repressing, delaying, or reducing metastasis of the tumor.

Exemplary cancer treatments include, without limitation, surgery, radiation, chemotherapies, anti-cancer biologics, immunotherapies, targeted cancer drugs (e.g., PARP inhibitors), stem cell therapies, and hormone therapies.

Examples of suitable chemotherapy agents include, but are not limited to, 5-fluorouracil, aclacinomycin, activated cytoxan, bisantrene, bleomycin, carmofur, CCNU, cis-platinum, daunorubicin, doxorubicin, DTIC, melphalan, methotrexate, mithromycin, mitomycin, mitomycin C, peplomycin pipobroman, plicamycin, procarbazine, retinoic acid, tamoxifen, taxol, tegafur, VP16, and VM25.

“Anti-cancer biologics” are biomolecules (e.g., polynucleotides, polypeptides, lipids, carbohydrates) that may be used to treat cancer. Examples of anti-cancer biologics include, but are not limited to, cytokines (e.g., IL-1α, IL-2, IL-2β, IL-3, IL-4, CTLA-2, IFN-α, IFN-γ, granulocyte-macrophage colony stimulating factor (GM-CSF), IL-12, IL-23, IL-15, IL-7) and anti-cancer antibodies (e.g., Rituximab, Trastuzumab, Gemtuzumab, Alemtuzumab, Ibritumomab tiuxetan, Tositumomab, Cetuximab, Bevacizumab, Panitumumab, Ofatumumab, Brentuximab Vedotin, Pertuzumab, Adotrastuzumab emtansine, Lapatinib, Erlotanib, Obinutuzumab).

“Immunotherapies” are agents that stimulate the immune system. Examples of immunotherapies include, but are not limited to, checkpoint inhibitors, cancer vaccines, engineered T cells, oncolytic viruses, and bispecific antibodies.

“Checkpoint inhibitors” are a type of immunotherapy that target immune checkpoints. Examples of checkpoint inhibitors include, without limitation, antibodies and other therapeutics that target programmed cell death protein 1 (PD1), programmed cell death 1 ligand 1 (PD-L1), PD-L2, cytotoxic T-lymphocyte antigen 4 (CTLA4), A2AR, CD27, CD28, CD40, CD80, CD86, CD122, CD137, OX40, GITR, ICOS, TIM-3, LAG3, B7-H3, B7-H4, BTLA, IDO, KIR, and VISTA. Suitable anti-PD1 antibodies include, without limitation, lambrolizumab (Merck MK-3475), nivolumab (Bristol-Myers Squibb BMS-936558), AMP-224 (Merck), and pidilizumab (CureTech CT-011). Suitable anti-PD-L1 antibodies include, without limitation, MDX-1105 (Medarex), MEDI4736 (Medimmune) MPDL3280A (Genentech/Roche), and BMS-936559 (Bristol-Myers Squibb). Exemplary anti-CTLA4 antibodies include, without limitation, ipilimumab (Bristol-Myers Squibb), and tremelimumab (Pfizer).

“Cancer vaccines” are vaccines that teach the immune system to recognize and destroy cancer cells. Cancer vaccines generally include a tumor antigen. Cancer vaccines include a variety of formulations, including, without limitation, dendritic cells, monocytes, viral, liposomal, and DNA vaccines. Exemplary cancer vaccines include, without limitation, Sipuleucel-T (Provenge®, or APC8015).

“Engineered T cells” are T cells that have been genetically modified to target cancer cells. Engineered T cells include “CAR T cells,” i.e., T cells T cells that have been genetically modified to express chimeric antigen receptor (CAR) proteins. CAR proteins may include a targeting moiety such as a single-chain variable fragment (scFv) that binds to a tumor-associated antigen, a transmembrane domain, and intracellular signaling/activation domain(s). The intracellular signaling/activation domain(s) may include, without limitation, CD3z signaling domains, 41BB-signaling domains, CD28-signaling domains, or combinations thereof. Suitable tumor-associated antigens include, without limitation, CD19, carcinoembryonic antigen (CEA), diganglioside GD2, mesothelin, L1 cell adhesion molecule (L1CAM), human epidermal growth factor receptor 2 (HER2), fibroblast activation protein (FAP), interleukin 13 receptor a (IL13Ra), epidermal growth factor receptor (EGFR), and EGFR variant 3 (EGFRvIII).

An “oncolytic virus” is a virus that preferentially infects and kills cancer cells. Examples of oncolytic viruses include, but are not limited to, PVS-RIPO, T-VEC, and Onyx-015.

A “bispecific antibody” is an antibody that can bind to two different antigens at the same time via two distinct binding domains. Bispecific antibodies that have one binding site for a tumor-associated antigen and one binding site for a T-cell surface receptor can be used to promote the lysis of tumor cells by T cells. Examples of suitable bispecific antibodies include, but are not limited to, Removab (Trion Pharma), Blincyto (Amgen), AMG-110 (Amgen), ABT-122 (Abbvie), ABT-981 (Abbvie), AFM13 (Affimed Therapeutics), MM-111 (Merrimack Pharmaceuticals), SAR156597 (Sanofi), RG7221 (Roche), RG6013 (Roche), RG7597 (Roche), ALX-0761 (Ablynx), MCLA-128 (Merus), MEDI-565 (AMG-211), MGD006 (Macrogenics), and REGN1979 (Regeneron).

### Compositions Comprising a Fragment of Mutant Desmin:

In a second aspect, the present invention provides compositions comprising a fragment of the mutant desmin protein of SEQ ID NO: 6 that comprises amino acid residue Y399 (i.e., the mutated residue that gets phosphorylated). The fragment compositions may contain peptide sequences derived from the mutant desmin protein that retain the phosphorylated tyrosine residue at position 399, enabling the fragments to interact with cellular targets in a manner similar to the full-length mutant protein. The fragment approach may offer advantages over full-length protein compositions by providing enhanced stability, improved bioavailability, and reduced immunogenicity while maintaining the functional characteristics associated with the phosphorylated tyrosine residue. The compositions may be designed to include specific amino acid sequences that encompass the mutation site and surrounding regions that contribute to protein function and cellular interactions. The fragment compositions may be formulated with pharmaceutically acceptable carriers to enhance delivery, stability, and therapeutic efficacy in clinical applications.

These mutant desmin protein fragments could potentially be used to treat cancer via two different mechanisms. First, they could potentially be used as decoy peptides that reduce the amount of phosphorylated mutant desmin protein in a tumor by swamping out the kinase responsible for phosphorylating it. The inventors hypothesize that phosphorylation of the mutant desmin protein may promote tumor metastasis. Thus, agents that reduce phosphorylation of this protein could potentially be used to reduce tumor metastasis.

Second, the mutant desmin protein fragments could potentially be used as anti-cancer vaccines that produce an immune response against cancer cells that expresses the mutant desmin protein. While desmin is a cytoskeletal protein that is not typically present on the cell surface, there is evidence in the literature that another intermediate filament protein (i.e., vimentin) can be used as an anti-cancer peptide vaccine (Ohara M et al. Phosphorylated vimentin as an immunotherapeutic target against metastatic colorectal cancer. Cancer Immunol Immunother 69(6): 989-999, 2020), which suggests that this strategy is feasible.

As used herein, a “fragment” is a portion of a protein that is identical in sequence to, but shorter in length than, the full-length protein. For example, a fragment of the mutant desmin polypeptide of SEQ ID NO: 6, which is 470 amino acids in length, may comprise at least 5, 10, 15, 20, 25, 30, 40, 50, 60, 70, 80, 90, 100, 150, 250, 300, 350, 400, 450 or 469 contiguous amino acid residues of the full-length protein. In some embodiments, the fragments are between 5 and 20 amino acids in length. A fragment may include an N-terminal truncation, a C-terminal truncation, or both an N-terminal and C-terminal truncation relative to the full-length protein. The fragments used in the compositions of the present invention may consist of any portion of the full-length mutant desmin protein so long as they include the mutated residue Y399. In the Examples, the inventors propose the use of the mutant desmin protein fragment of SEQ ID NO: 3 (MALYVEIATYR), which was identified via mass spectrometry after trypsin digestion of the protein and comprises the mutation. Thus, in some embodiments, the compositions comprise or consist of the peptide of SEQ ID NO: 3. In some embodiments the peptide may comprise SEQ ID NO: 3 and include no more than 20, 25 or 30 amino acids. Longer fragments approaching 20 amino acids may provide extended sequence context that more closely resembles the native protein environment surrounding the mutation site, potentially enhancing biological activity and target specificity. The optimal fragment length may be determined through systematic evaluation of biological activity, stability characteristics, and therapeutic efficacy across the specified size range. The fragment length selection may also consider manufacturing feasibility and cost considerations associated with peptide synthesis and purification processes. The sequence may be synthesized using standard peptide synthesis techniques that enable incorporation of phosphorylated amino acids during the assembly process or may be generated using recombinant biology techniques via expression from a polynucleotide encoding the peptide.

The protein fragments described herein may be altered to make them more stable, i.e., for improved shelf-life and/or in vivo half-life. Examples of suitable methods for stabilizing peptides include (a) cyclization (i.e., the joining the termini of a peptide to produce a circular peptide), (b) dimerization, (c) substitution of natural amino acids with non-natural amino acids (e.g., replacement of the cysteine with homocysteine or α-methyl-cysteine), (d) substitution of L-amino acids with D-amino acids, (e) alteration of peptide bonds (e.g., addition of a methyl group), (f) addition of non-amino acid moieties (e.g., polyethylene glycol (PEG), acetyl group, carbamyl group, formyl group, myristoyl group) to the N- or C-terminus, and (g) fusion to a carrier protein.

In some embodiments, the compositions further comprise a pharmaceutically acceptable carrier. The term “pharmaceutically acceptable carrier” refers to any carrier, diluent, or excipient that is compatible with the other ingredients of a formulation and is not deleterious to a recipient to which it is to be administered. Pharmaceutically acceptable carriers that enhance peptide stability, facilitate cellular delivery, and improve therapeutic outcomes through optimized pharmacokinetic properties may be used. Pharmaceutically acceptable carriers may include excipients, delivery vehicles, and formulation components that have been approved for human use and demonstrate compatibility with peptide-based therapeutics. The carrier selection may be based on the intended route of administration, target tissue distribution, and desired release characteristics for the therapeutic application. The pharmaceutically acceptable carriers may provide protection against enzymatic degradation, enhance membrane permeability, and facilitate targeted delivery to specific cell types or anatomical locations. The carrier systems may be designed to maintain peptide integrity during storage, transport, and administration while providing controlled release characteristics that optimize therapeutic efficacy and minimize adverse effects. Pharmaceutically acceptable carriers are known in the art and include, but are not limited to, diluents (e.g., Tris-HCl, acetate, phosphate), preservatives (e.g., thimerosal, benzyl alcohol, parabens), solubilizing agents (e.g., glycerol, polyethylene glycerol), emulsifiers, liposomes, nanoparticles, and adjuvants. Pharmaceutically acceptable carriers may be aqueous or non-aqueous solutions, suspensions, or emulsions.

In some embodiments, the protein fragments are packaged into delivery particles, such as nanoparticles, extracellular vesicles, or liposomes, for improved in vivo delivery. A “nanoparticle” is a particle that is 1-100 nm in diameter. An “extracellular vesicle” (EV) is a small, membrane-bound particle that is released from a cell. A “liposome” is a small, spherical, artificial vesicle with at least one lipid bilayer that mimics a cell membrane structure. The delivery particles may be configured to include a ligand (e.g., an antibody) that specifically targets a particular cell type (e.g., a particular cancer cell surface marker). The delivery particles may contain the protein fragments or be linked via a linker to the protein fragments. The linker may be a peptide linker.

The therapeutic formulations may be specifically designed for treating pancreatic ductal adenocarcinoma through targeted delivery of the mutant desmin protein fragments to tumor tissues and associated stromal components. The formulation development may consider the unique characteristics of pancreatic tumors including dense desmoplastic stroma, limited vascular access, and elevated interstitial pressure that may impede drug delivery. The carrier systems may be optimized to enhance penetration through fibrotic tissue barriers and achieve adequate drug concentrations within tumor microenvironments. The therapeutic formulations may incorporate penetration enhancers, matrix metalloproteinase-cleavable linkers, or other strategies that facilitate drug distribution throughout pancreatic tumor masses. The formulation stability may be optimized for storage conditions and administration requirements associated with clinical use in pancreatic cancer treatment protocols. The therapeutic compositions may be designed for compatibility with existing treatment regimens including chemotherapy, radiation therapy, or immunotherapy approaches that may be used in combination with the peptide-based therapeutics.

The fragment stabilization approaches may enhance the therapeutic utility of the mutant desmin protein compositions through modifications that improve peptide stability, bioavailability, and resistance to enzymatic degradation. Cyclization techniques may involve formation of disulfide bonds, lactam bridges, or other covalent linkages that constrain peptide conformation and reduce susceptibility to proteolytic cleavage. The cyclization process may utilize cysteine residues introduced at strategic positions within the peptide sequence to form intramolecular disulfide bonds that stabilize secondary structure elements. Alternative cyclization approaches may employ non-natural amino acids containing reactive side chains that enable formation of stable covalent linkages through chemical crosslinking reactions. The cyclized peptides may exhibit enhanced resistance to exopeptidase and endopeptidase activities that typically limit the half-life of linear peptides in biological systems. The cyclization strategy may be designed to preserve the functional conformation of the phosphorylated tyrosine residue and surrounding amino acids while providing overall structural stabilization.

Dimerization strategies may involve linking two copies of the mutant desmin protein fragment through covalent or non-covalent interactions that enhance binding affinity, increase biological activity, and improve pharmacokinetic properties. The dimerization process may utilize chemical crosslinking agents such as glutaraldehyde, carbodiimides, or bifunctional polyethylene glycol derivatives that form stable linkages between amino acid side chains on separate peptide molecules. Alternative dimerization approaches may employ genetically encoded dimerization domains, leucine zippers, or other protein-protein interaction motifs that promote spontaneous association of peptide fragments. The dimeric structures may exhibit enhanced target binding through avidity effects that result from simultaneous engagement of multiple binding sites on target proteins or cellular receptors. The dimerization may also provide increased resistance to proteolytic degradation by creating larger molecular structures that may be less accessible to proteolytic enzymes. The dimeric peptides may demonstrate improved pharmacokinetic profiles including reduced renal clearance and extended circulation times compared to monomeric fragments.

The incorporation of non-natural amino acids may provide additional stabilization mechanisms that enhance the therapeutic properties of the mutant desmin protein fragments while maintaining biological activity. Non-natural amino acids may include D-amino acid analogs, amino acids with modified side chains, or synthetic amino acid derivatives that confer resistance to enzymatic degradation or altered binding characteristics. The substitution of L-amino acids with D-amino acids may create peptides that are resistant to most naturally occurring proteases while retaining the ability to interact with target proteins or cellular receptors. The D-amino acid substitutions may be strategically positioned within the peptide sequence to maximize stability benefits while minimizing disruption of functional domains such as the phosphorylated tyrosine residue at position 399. The non-natural amino acid modifications may include incorporation of fluorinated amino acids, amino acids with bulky side chains, or amino acids containing reactive functional groups that enable additional chemical modifications. The selection of non-natural amino acids may be guided by computational modeling studies that predict the effects of amino acid substitutions on peptide conformation and target binding affinity.

Peptide bond alterations may provide alternative stabilization approaches that modify the backbone structure of the mutant desmin protein fragments to enhance resistance to proteolytic degradation while preserving biological function. The peptide bond modifications may include incorporation of peptide bond isosteres such as reduced amide bonds, thioamide bonds, or ester linkages that are not recognized by conventional proteases. The backbone modifications may be introduced at specific positions within the peptide sequence that are susceptible to proteolytic cleavage based on known protease specificity patterns. The altered peptide bonds may maintain the overall three-dimensional structure of the peptide while providing enhanced stability in biological environments. The peptide bond modifications may be combined with other stabilization strategies to achieve synergistic effects that maximize peptide half-life and therapeutic efficacy. The selection of peptide bond alterations may consider the synthetic accessibility of the modified peptides and the compatibility of the modifications with large-scale manufacturing processes.

The addition of non-amino acid moieties may provide further stabilization and functional enhancement of the mutant desmin protein fragments through conjugation with polymers, lipids, or other chemical entities that modify peptide properties. Polyethylene glycol conjugation may increase peptide molecular weight, reduce immunogenicity, and extend circulation half-life through reduced renal clearance and proteolytic degradation. The polyethylene glycol attachment may be accomplished through reaction with amino acid side chains, N-terminal amino groups, or C-terminal carboxyl groups using established conjugation chemistries. Lipid conjugation may enhance membrane association and cellular uptake of the peptide fragments while providing protection against enzymatic degradation. The lipid moieties may include fatty acids, phospholipids, or cholesterol derivatives that facilitate membrane insertion and intracellular delivery. Other non-amino acid modifications may include attachment of fluorescent labels for tracking purposes, radioactive isotopes for imaging applications, or targeting ligands that enhance selective delivery to specific cell types or tissues.

Fusion to carrier proteins may provide comprehensive stabilization and delivery enhancement for the mutant desmin protein fragments through association with larger protein structures that offer multiple functional benefits. The carrier proteins may include albumin, immunoglobulin fragments, or engineered protein scaffolds that provide extended circulation times, reduced immunogenicity, and enhanced tissue distribution properties. The fusion process may involve genetic engineering approaches that create recombinant proteins containing both the carrier protein and the mutant desmin fragment as a single polypeptide chain. Alternative fusion strategies may utilize chemical conjugation methods that link the peptide fragment to carrier proteins through covalent bonds formed between reactive amino acid side chains. The carrier protein selection may consider factors such as biocompatibility, manufacturing feasibility, and compatibility with the intended therapeutic application. The fusion proteins may be designed to include cleavable linkers that enable release of the active peptide fragment at target sites through enzymatic or chemical cleavage mechanisms. The carrier protein fusions may provide opportunities for incorporating additional functional domains such as cell-penetrating peptides, targeting sequences, or therapeutic moieties that enhance the overall therapeutic profile of the composition.

### Polynucleotides:

In a third aspect, the present invention provides polynucleotides encoding a mutant desmin protein fragment disclosed herein.

The terms “polynucleotide,” “nucleic acid,” and “oligonucleotide” are used interchangeably to refer a polymer of DNA or RNA. A polynucleotide may be single-stranded or double-stranded and may represent the sense or the antisense strand. A polynucleotide may be synthesized or obtained from a natural source. A polynucleotide may contain natural, non-natural, or altered nucleotides, as well as natural, non-natural, or altered internucleotide linkages (e.g., phosphoroamidate linkages, phosphorothioate linkages). In some embodiments the polynucleotide may be an mRNA capable of translation to form the desmin protein or a fragment thereof.

The polynucleotides of the present invention may be codon-optimized for expression in a particular cell type. “Codon optimization” is a process used to increase expression of a polynucleotide in a particular host cell by altering the sequence of the polynucleotide to accommodate the codon bias of the host cell. Computer programs for generating codon-optimized sequences for use in a particular host cell are known in the art.

In some embodiments, the polynucleotide is part of a construct. As used herein, the term “construct” refers a to recombinant polynucleotide, i.e., a polynucleotide that was formed by combining at least two polynucleotide components from different sources. For example, a construct may comprise the coding region of one gene operably linked to a promoter that is (1) associated with another gene found within the same genome, (2) from the genome of a different species, or (3) synthetic. Constructs can be generated using conventional recombinant DNA methods. In some embodiments, the construct further comprises a promoter that is operably linked to the polynucleotide including the mutant desmin protein fragment. As used herein, the term “promoter” refers to a DNA sequence that defines where transcription of a polynucleotide begins. RNA polymerase and the necessary transcription factors bind to the promoter to initiate transcription. Promoters are typically located directly upstream (i.e., at the 5′ end) of the transcription start site. However, a promoter may also be located at the 3′ end, within a coding region, or within an intron of a gene that it regulates. Promoters may be derived in their entirety from a native or heterologous gene, may be composed of elements derived from multiple regulatory sequences found in nature, or may comprise synthetic DNA. A promoter is “operably linked” to a polynucleotide if the promoter is positioned such that it can affect transcription of the polynucleotide. Suitable promoters for use with the present invention include, but are not limited to, constitutive, inducible, temporally regulated, developmentally regulated, chemically regulated, tissue-preferred, and tissue-specific promoters. The promoter may be an animal, plant, bacterial, fungal, or synthetic promoter.

Polynucleotide constructs may provide genetic delivery systems for expressing mutant desmin protein fragments within target cells through the introduction of nucleic acid sequences that encode specific portions of the mutant desmin protein. The constructs may comprise polynucleotides encoding at least a portion of SEQ ID NO: 6 operably connected to a promoter, wherein the portion of SEQ ID NO: 6 comprises at least 8 amino acids and includes amino acid 399Y. The polynucleotide sequences may be designed to encode peptide fragments that retain the functional characteristics associated with the D399Y mutation while providing manageable genetic constructs for cellular delivery and expression. The constructs may utilize various promoter systems to control the timing, location, and level of mutant desmin protein fragment expression within target cells. The polynucleotide approach may offer advantages over direct peptide delivery by enabling sustained production of therapeutic fragments within cells and providing opportunities for tissue-specific or inducible expression patterns.

In some embodiments, the construct is a vector. The term “vector” refers to a DNA molecule that is used to carry a particular DNA segment into a host cell. Some vectors are capable of autonomous replication in a host cell (e.g., bacterial vectors that include an origin of replication and episomal mammalian vectors). Other vectors can be integrated into the genome of a host cell such that they are replicated along with the host genome (e.g., viral vectors and transposons). Vectors may include heterologous genetic elements that are necessary for propagation of the vector or for expression of an encoded gene product. Vectors may also include a reporter gene or a selectable marker gene. Suitable vectors include plasmids (i.e., circular double-stranded DNA molecules) and mini-chromosomes.

In some embodiments, the vector is a gene therapy vector. As used herein, a “gene therapy vector” is a vector used to deliver a polynucleotide into cells within a subject. Delivery of a polynucleotide encoding a mutant desmin protein fragment could allow the fragment to be overexpressed in a tumor microenvironment, which could reduce phosphorylation of any mutant desmin protein expressed by the tumor and thereby reduce the risk of metastasis.

### Methods of Treating Cancer Using a Fragment of Mutant Desmin:

In a fourth aspect, the present invention provides methods of treating a cancer in a subject. The methods comprise administering a composition or polynucleotide described herein to the subject. The treatment methods may utilize the unique properties of the mutant desmin protein fragments to disrupt cancer cell functions while minimizing effects on normal cellular processes. The therapeutic approach may be based on the specific presence of the phosphorylated D399Y mutation in cancer cells, providing a molecular target that distinguishes malignant tissues from healthy tissues. The treatment methods may be applied to various cancer types, with particular applicability to pancreatic ductal adenocarcinoma where the mutant desmin protein has been identified as a characteristic molecular feature. The administration of mutant desmin protein fragments may provide therapeutic benefits through multiple mechanisms including competitive inhibition of cellular processes and immune system activation against cancer cells expressing the mutant protein.

The treatment methods may involve administering compositions to subjects who have cancer expressing a mutant desmin protein comprising a phosphorylated D399Y mutation, providing targeted therapy for patients with specific molecular characteristics. The identification of subjects with cancer expressing the mutant desmin protein may be accomplished through diagnostic testing using the detection methods described herein, enabling patient stratification based on molecular tumor features. As used herein, the term “administering” refers to the introduction of a substance into a subject's body. Methods of administration are well known in the art and include, but are not limited to, oral administration, transdermal administration, administration by inhalation, nasal administration, topical administration, intravaginal administration, ophthalmic administration, intraoral administration, intracerebral administration, rectal administration, sublingual administration, buccal administration, and parenteral administration, including injectable such as intravenous administration, intra-arterial administration, intramuscular administration, intradermal administration, intrathecal administration, and subcutaneous administration. Administration can be performed in multiple doses and may be continuous or intermittent.

Any form of cancer that expresses a mutant desmin protein comprising a phosphorylated D399Y mutation may be treated using the present methods. However, in the Examples, the inventors identified this mutant desmin protein in PDAC samples. Thus, in preferred embodiments, the cancer is PDAC.

Without being limited by theory, the therapeutic mechanism may involve the use of mutant desmin protein fragments as decoy peptides that reduce the amount of phosphorylated mutant desmin protein in tumors by competing with kinases responsible for phosphorylation. The decoy peptide approach may exploit the substrate specificity of cellular kinases that normally phosphorylate the mutant desmin protein at the tyrosine 399 residue. The therapeutic fragments may serve as competitive substrates that bind to kinase active sites and undergo phosphorylation, thereby reducing the availability of kinase activity for phosphorylating the endogenous mutant desmin protein. The competition for kinase activity may result in decreased phosphorylation of the mutant desmin protein within cancer cells, potentially disrupting cellular processes that depend on the phosphorylated form of the protein. The decoy mechanism may be particularly effective against Src family kinases or other tyrosine kinases that have been implicated in phosphorylating the mutant desmin protein. The therapeutic fragments may be designed with enhanced kinase binding affinity compared to the endogenous mutant desmin protein, enabling effective competition even at relatively low therapeutic concentrations.

The compositions provided herein may also be used as an anti-cancer vaccine. This application may involve formulation of the mutant desmin protein fragments with adjuvants or delivery systems that enhance immune recognition and response generation. The vaccine compositions may include immunostimulatory agents such as toll-like receptor agonists, cytokines, or other immune modulators that promote antigen presentation and T-cell activation. The therapeutic fragments may be conjugated to carrier proteins or incorporated into delivery vehicles that facilitate uptake by dendritic cells and other antigen-presenting cells. The vaccine approach may involve multiple administrations to achieve optimal immune priming and memory formation, with booster doses provided to maintain therapeutic immune responses over extended periods. The immunization schedule may be optimized based on immune response kinetics and the persistence of therapeutic effects following vaccination. The vaccine strategy may be combined with checkpoint inhibitors or other immunotherapeutic agents to enhance immune activation and overcome tumor-associated immune suppression mechanisms

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference unless explicitly indicated otherwise. The present disclosure shall control in the event there are any disparities between any definitions and/or descriptions found in the cited references.

The following examples are meant only to be illustrative and are not meant as limitations on the scope of the invention or of the appended claims.

### EXAMPLES

### Introduction

Pancreatic ductal adenocarcinoma (PDAC) is a deadly cancer characterized by a high percentage of KRAS mutations, extensive desmoplasia, and rapid metastasis with a 5-year survival rate of 12% [1, 2]. Desmoplasia is a formation of dense fibrotic tissue containing myofibroblasts, pancreatic stellar cells (PSC), and tumor cells comprising 50-80% by volume of the PDAC tumor. In most patients it probably begins when mutant KRAS in tumor cells triggers the release of cytokines, chemokines, and growth factors that activate nearby quiescent PSCs causing them to release collagens and other stromal proteins [3]. Yes-associated protein (YAP), a transcriptional co-activator and effector of the Hippo signaling pathway, is also involved [4], as are Src family tyrosine kinases (SFKs) [5]. Interactions between PSC and tumor cells exacerbate desmoplasia to the point where high interstitial pressure causes vascular collapse that prevents drug entry [3]. Thus, desmoplasia itself is a PDAC drug target [6].

Our laboratory has shown that phosphotyrosine western blotting (pTyr WB) is sensitive enough to detect activated receptor tyrosine kinase (pTyr-RTK) driver proteins in lung squamous cell carcinoma [7, 8]. Since SFKs, KRAS, and YAP are involved in PDAC, and since wild-type pTyr-RTKs might be unknown drivers, we decided to use western blotting to probe for these proteins in PDAC tumor tissues purchased from a biobank. If a promising pTyr-RTK driver or substrate for a SFK driver of PDAC were found, then, after verification, an antibody against the pTyr-protein biomarker could be used for immunohistochemistry (IHC) testing of human biopsies to help guide drug treatment. Many TK inhibitors are now available [9].

We began by comparing western blot patterns of six PDAC tumor and five normal adjacent tissue (NAT) samples using antibodies against KRAS, YAP, Src, and pTyr-protein. Two of the six PDAC tumors (i.e., P1 and P2) showed abundant pTyr-protein bands at about 60 kDa as well as dark KRAS and YAP1 bands relative to NAT controls. To identify the ˜60 kDa pTyr-protein(s), we performed 2D pTyr WB of the P1 and P2 samples with matching 2D Coomassie stained gels to obtain enough purified stained protein for analysis by mass spectrometry (MS). The ˜60 kDa protein was identified as pTyr-mutant desmin with the mutation being aspartate 399 mutated to tyrosine 399 (D399Y). The pTyr residue was identified as mutant Y399. Possible consequences and ramifications of this surprising and potentially important observation are discussed below.

### Results:

All tumor and NAT tissue samples were dissolved in SDS buffer with protease and phosphatase inhibitors, as described in methods, to ensure complete protein recovery. All MS was performed on 1D bands or 2D spots cut from sodium dodecylsulfate polyacrylamide electrophoresis (SDS PAGE) gels [10].

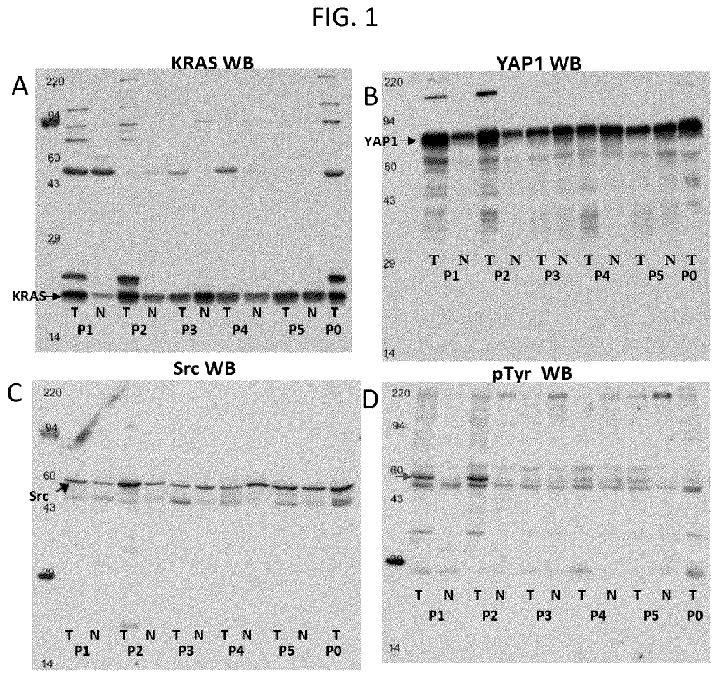

FIG. 1 shows western blots for KRAS, YAP, Src, and pTyr from four one-dimensional (1D) gels loaded with 40 μg total protein/lane from five alternating PDAC tumor/NAT samples and a singleton tumor sample (i.e., P0-T) from an earlier date. Samples P1 and P2 showed patterns similar to each other and different from the rest of the samples. The results presented in this figure are summarized below:
FIG. 1A: The KRAS western blot showed that the 21 kDa KRAS protein was strongly expressed in most of the samples and was clearly more abundant in P1 and P2 tumors versus controls, in contrast to the P3, P4, and P5 tumor samples. (P0 had no control.) Samples P1-T, P2-T, and P0-T showed several higher MW bands in the KRAS western blot, including a dark band at ˜24 kDa of unknown identity. We speculated these might be Ras covalent binding partners or Ras nanoclusters [11], but there was not enough material for identification. (The latter would require affinity resin purification before MS analysis.)
FIG. 1B: The YAP1 western blot showed that YAP1 was strongly expressed in most of the samples and was clearly more abundant in P1 and P2 tumors versus controls.
FIG. 1C: The Src western blot revealed 60 kDa bands were a little darker in the P1 and P2 tumor samples versus NAT controls, but not dramatically so. These bands were roughly the same intensity for the P3, P4, and P5 tumor samples as compared to controls.
FIG. 1D: The pTyr western blot showed a relatively dark pTyr protein band near the 60 kDa marker in the P1 and P2 tumor samples that was absent from control samples (red arrow).

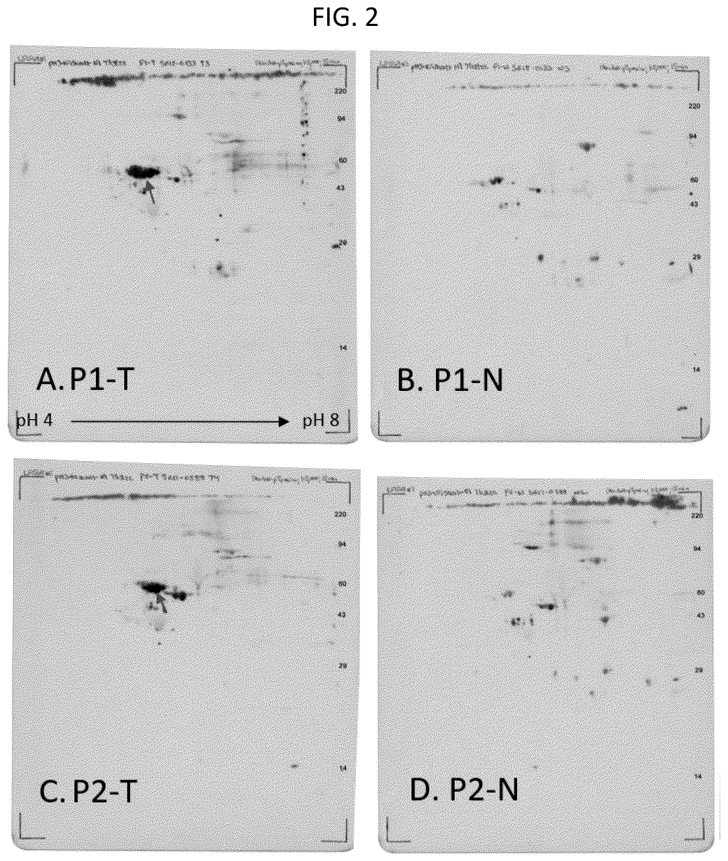

Since tyrosine phosphorylation is important for many cancer cell processes [12, 13], we proceeded with identification of the unknown ˜60 kDa pTyr-protein using purified protein cut from 2D gels. The next round of analysis employed two-dimensional gel electrophoresis (2DE) [10] in combination with pTyr western blots and Coomassie staining of samples P1 and P2. FIG. 2 shows 2D pTyr western blots of P1-Tumor (FIG. 2A), P1-NAT (FIG. 2B), P2-Tumor (FIG. 2C), and P2-NAT (FIG. 2D).

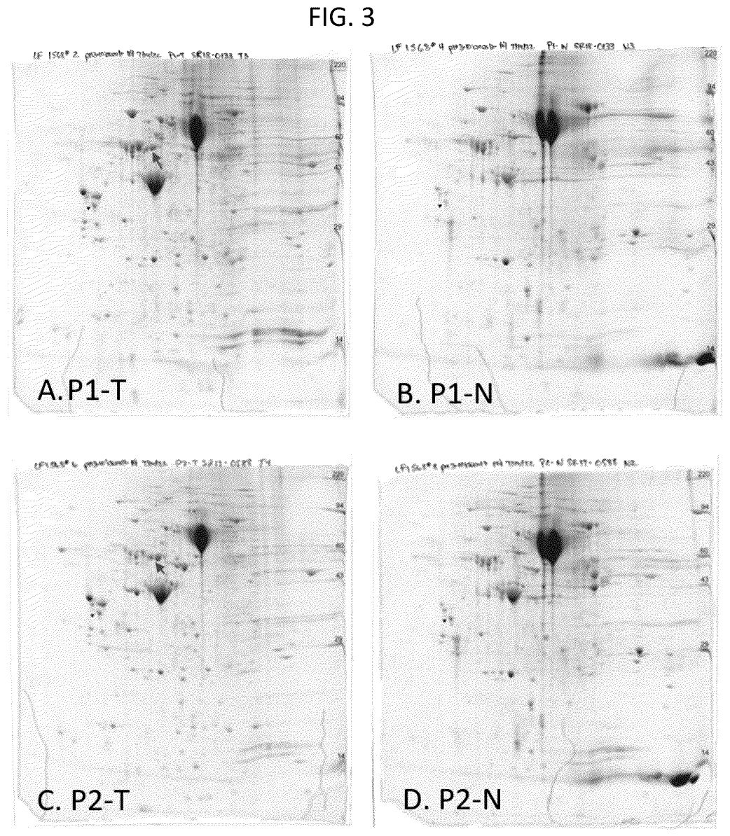

FIG. 3 shows matching Coomassie-stained gels showing total protein patterns in these two samples and their controls. Red arrows mark the ˜60 kDa pTyr-protein of interest, when present. Note that basic proteins with isoelectric points >8, are not visible on these 2D gels.

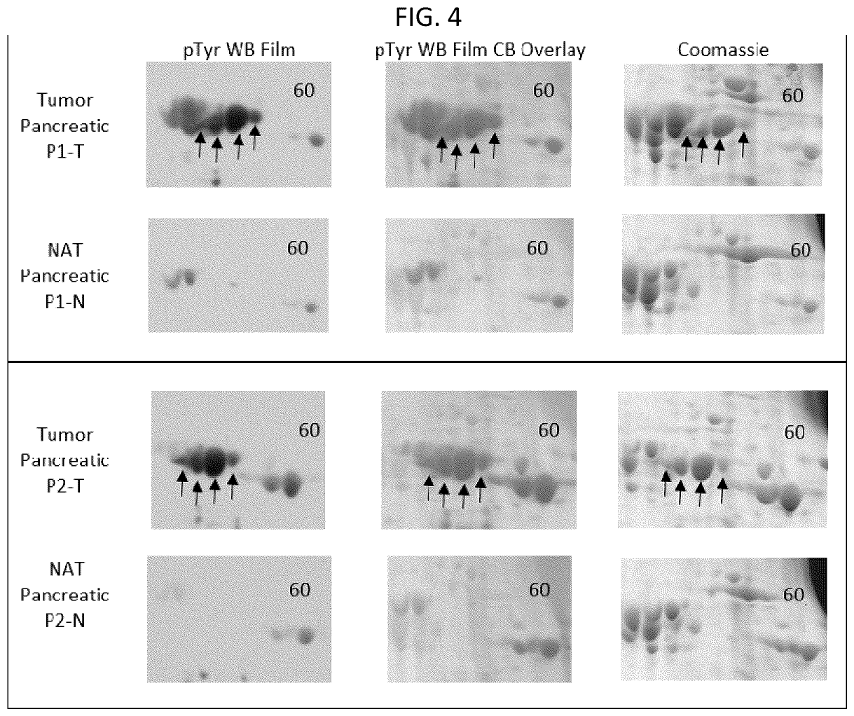

FIG. 4 shows a closeup of the ˜60 kDa pTyr-protein(s) first identified by 1D pTyr western blot in FIG. 1, then resolved by 2D pTyr western blot in FIG. 2 and by 2D Coomassie blue staining in FIG. 3. The middle overlay images of western blots over Coomassie-stained gels were used to guide Coomassie spot cutting for MS. The ˜60 kDa pTyr unknown protein resolves as a cluster of 4 isoforms (arrows), which are missing or very faint in the NAT samples.

To determine if the four protein spots were different proteins or isoforms of the same protein, we first cut out each of the four spots from a single Coomassie stained 2D gel for individual identification by nano liquid chromatography tandem mass spectrometry (NanoLC-MS/MS). All the isoforms were identified as mutant desmin, MW 55 kDa. Charge isoform clusters such as this are common on 2D gels and likely due to post-translational modifications such as cysteine oxidation, carbamylation, glycosylation, or phosphorylation that may change the isoelectric point of a protein [14, 15].

Since the pTyr post-translational modification (PTM) is rare and often difficult to identify [16], we used 2DE to extract purified desmin protein from a relatively large amount of tumor homogenate to identify the phosphorylated tyrosine residue as well as the desmin mutation. To do so we ran 500 μg of each of the P1-T and P2-T samples on three large-format 2D gels (1500 μg total). We estimated that relative to our internal standard, each pTyr-protein cluster (including 4 isoforms combined) was >2 μg of protein. Finally, we excised the pTyr-protein 4-spot clusters in FIG. 4 from the six 2D gels and combined them (>12 ug) for analysis by NanoLC-MS/MS [17].

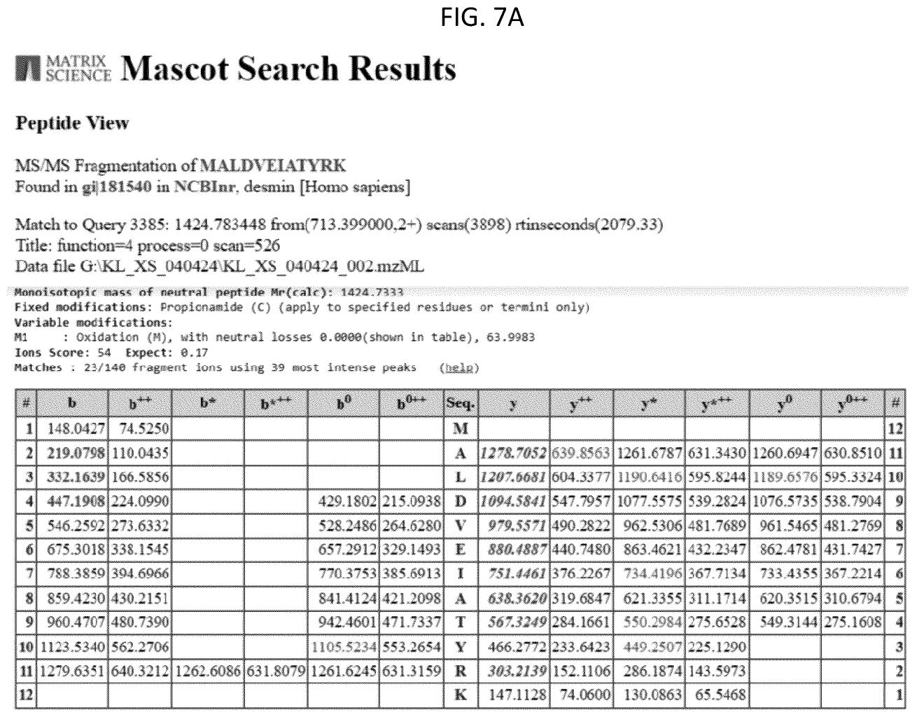

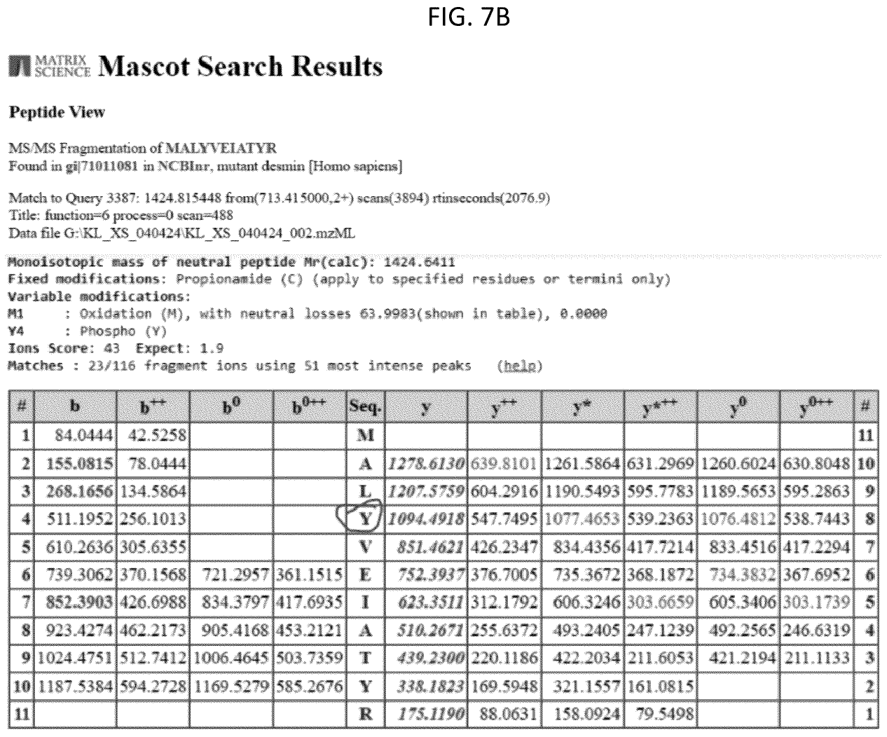

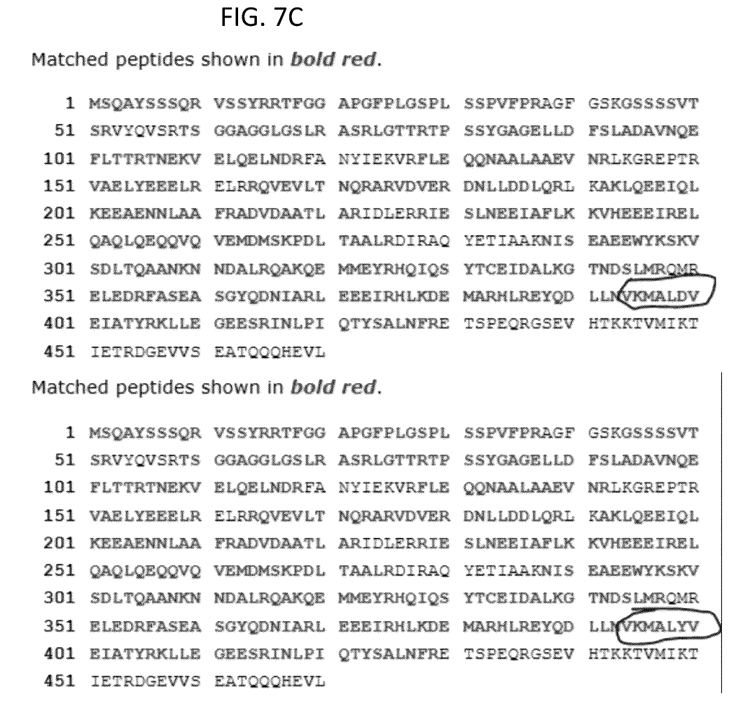

NanoLC-MS/MS analysis on a relatively large scale led to a surprising discovery: in addition to wild-type desmin [Homo sapiens](gi|181540; FIG. 7A), we also found a mutant desmin [Homo sapiens](gi|71011081; FIG. 7B). While amino acids 396-407 of wild-type desmin are MALDVEIATYRK (SEQ ID NO: 2; aspartate 399 is highlighted with bold font), amino acids 396-407 of the novel mutant protein are MALYVEIATYR (SEQ ID NO: 3; the D399Y mutation is highlighted with bold font). The peptide from the wild-type desmin protein was identified by precursor ions with m/z of 470.59 (3+) and 705.41 (2+) that corresponded to peptide MALDVEIATYRK (SEQ ID NO: 2), and by precursor ions with m/z of 475.93 (3+) and 713.41 (2+), which corresponded to peptide mALDVEIATYRK (SEQ ID NO: 2), wherein the lowercase “m” represent oxidized methionine.

Conversely, the peptide from the mutant desmin was identified by the precursor ions with m/z of 470.59 (3+) and 705.41 (2+) that corresponded to peptide MALpYVEIATYR (SEQ ID NO: 4; pY: phosphorylated tyrosine) and by precursor ions with m/z of 475.93 (3+) and 713.41 (2+), which corresponded to peptide mALpYVEIATYR (SEQ ID NO: 4).

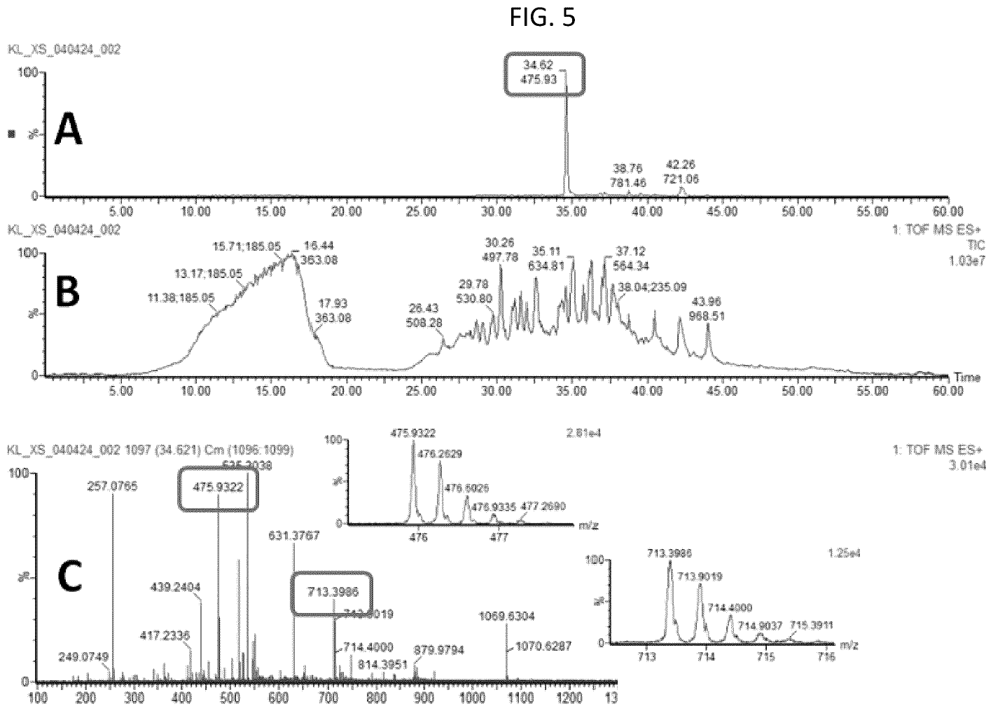

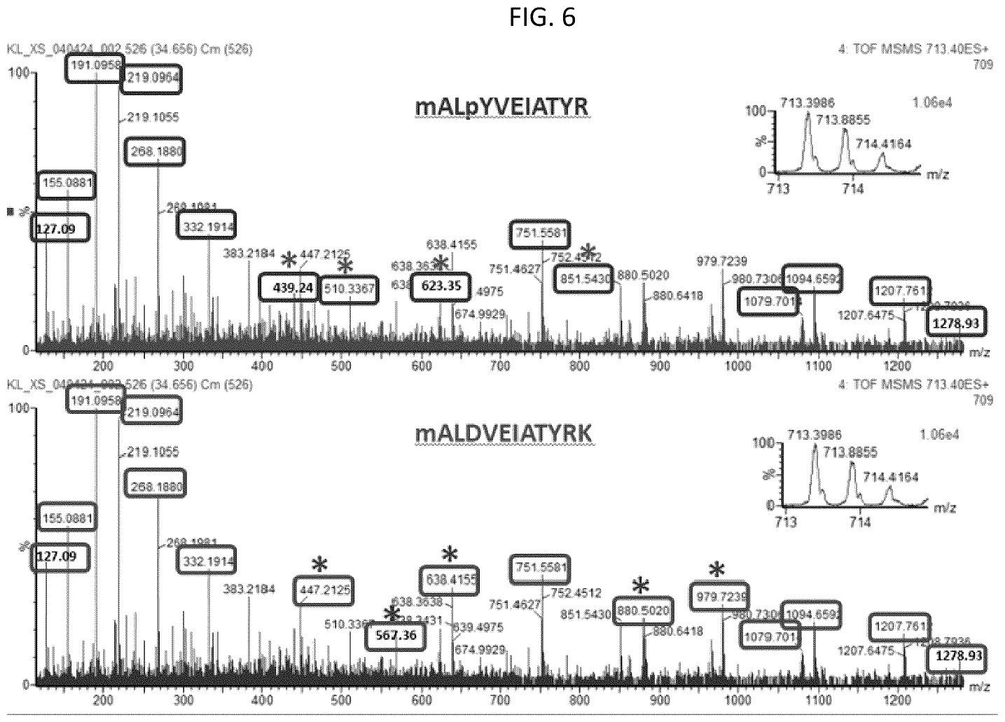

The MS and MS/MS spectra that correspond to peptides mALDVEIATYRK (SEQ ID NO: 2) from canonical desmin protein and mALpYVEIATYRK (SEQ ID NO: 5) from the mutant desmin protein are shown in FIG. 5 and FIG. 6. Unphosphorylated mutant peptide MALYVEIATYR (SEQ ID NO: 3) was not detected.

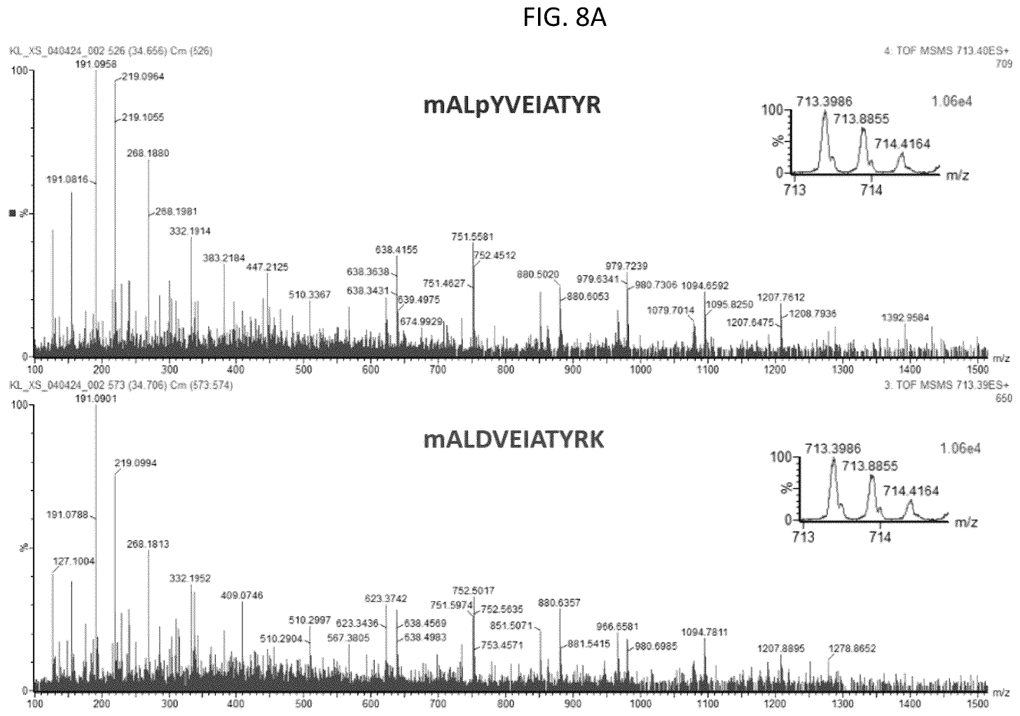

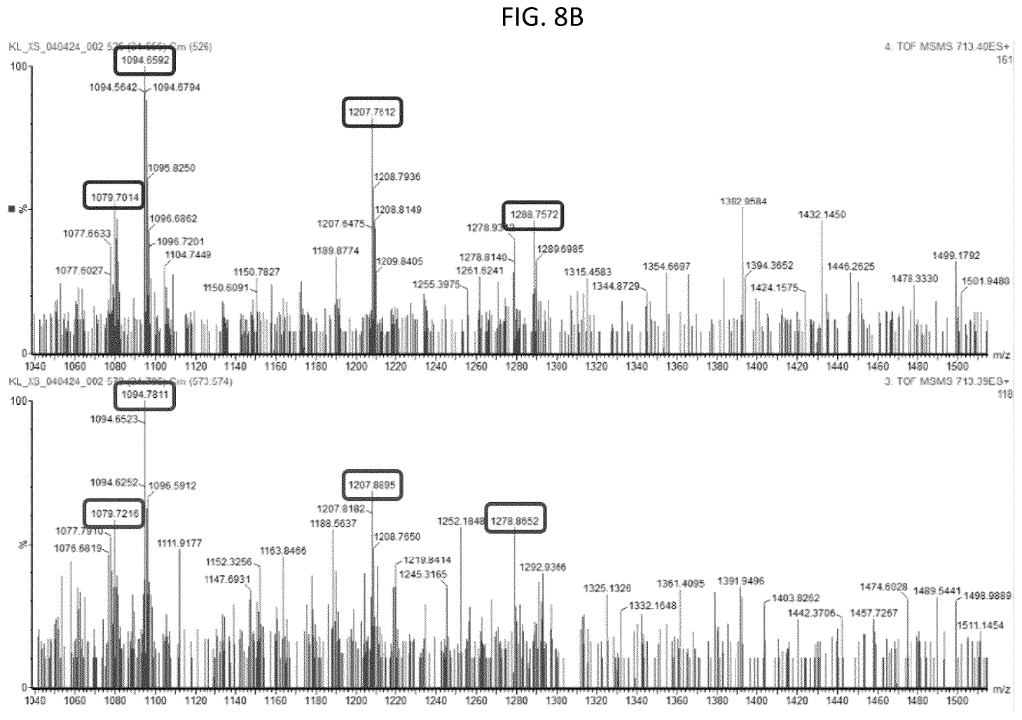

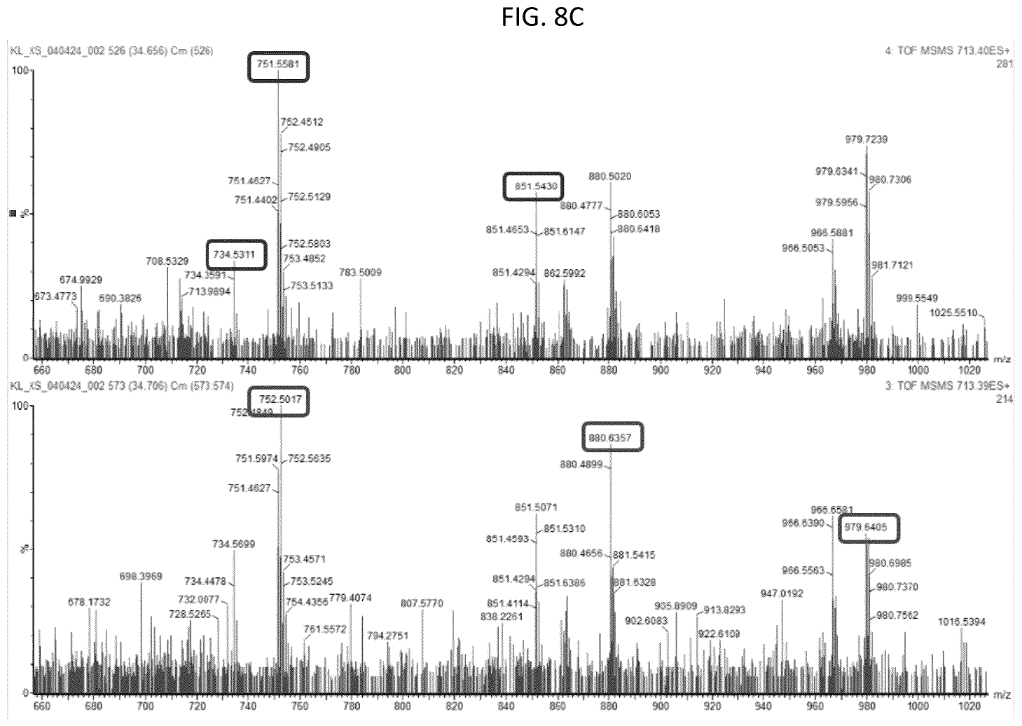

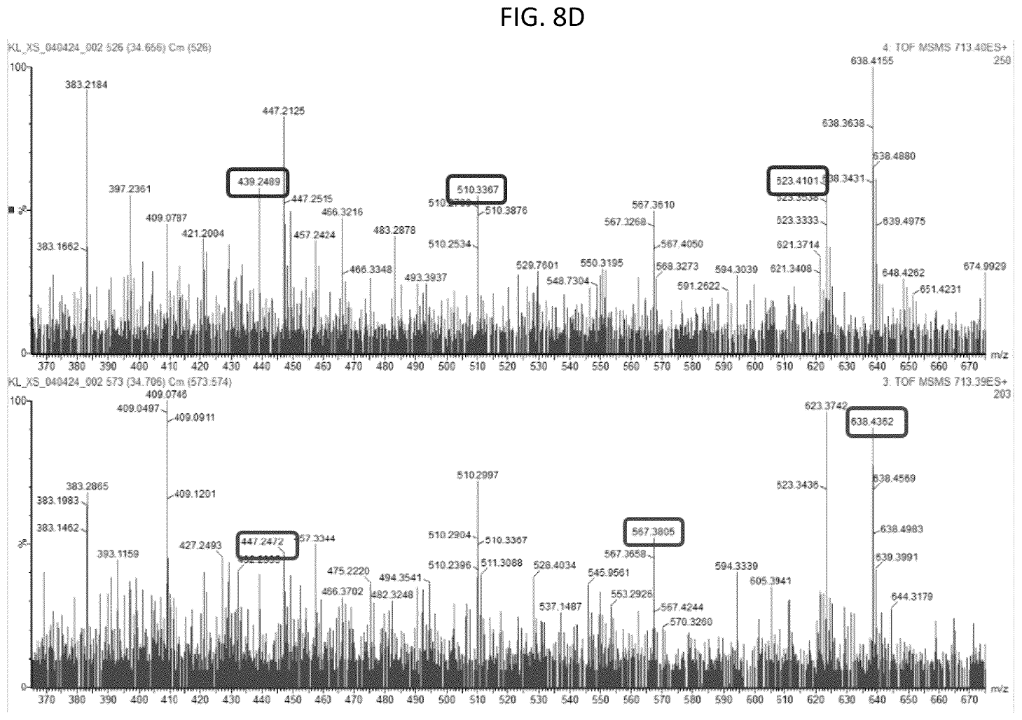

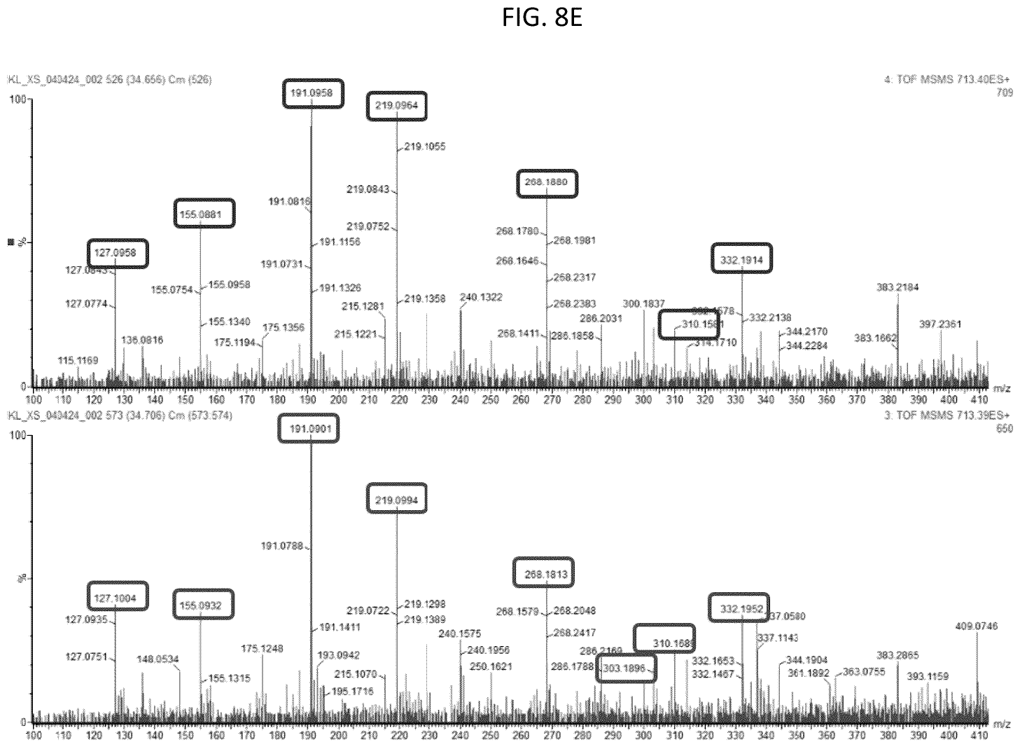

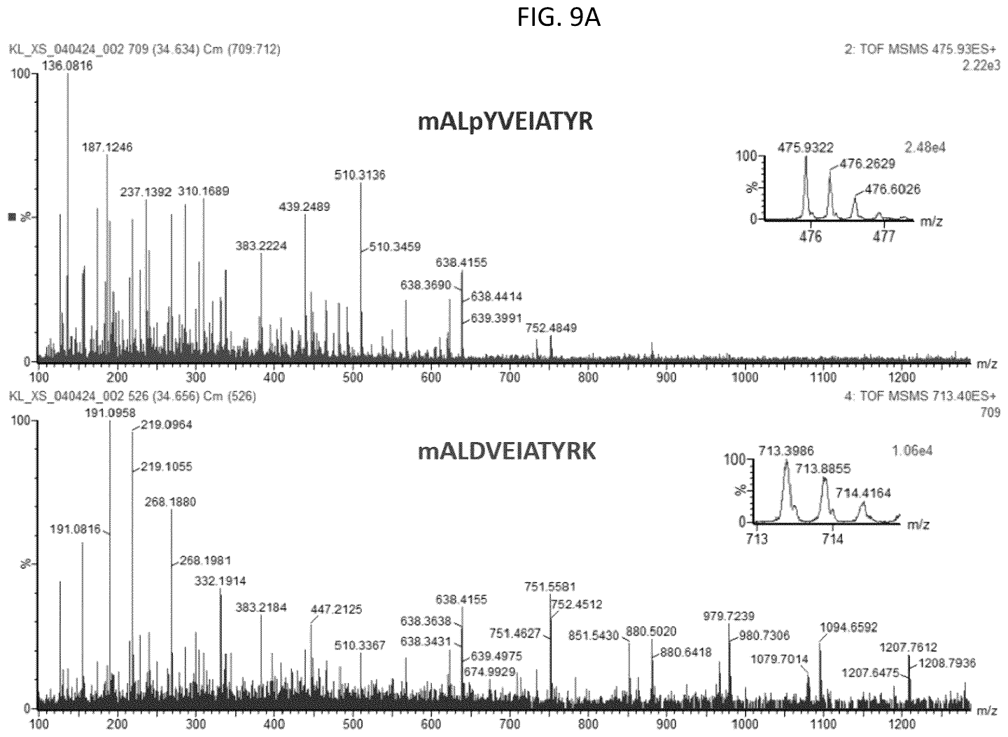

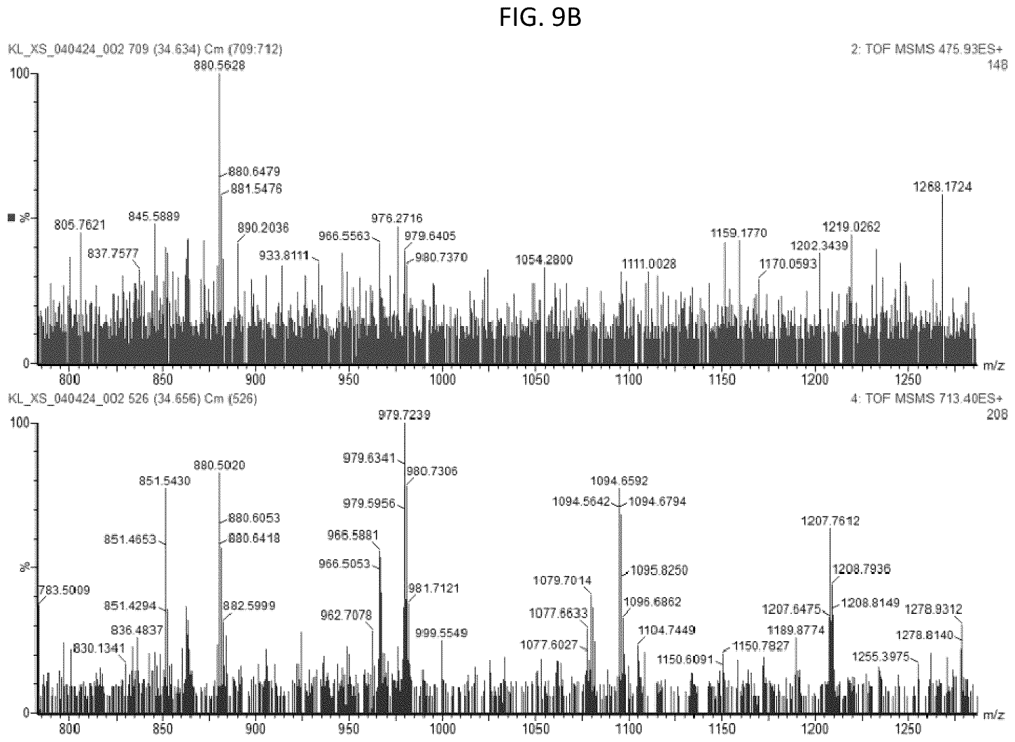

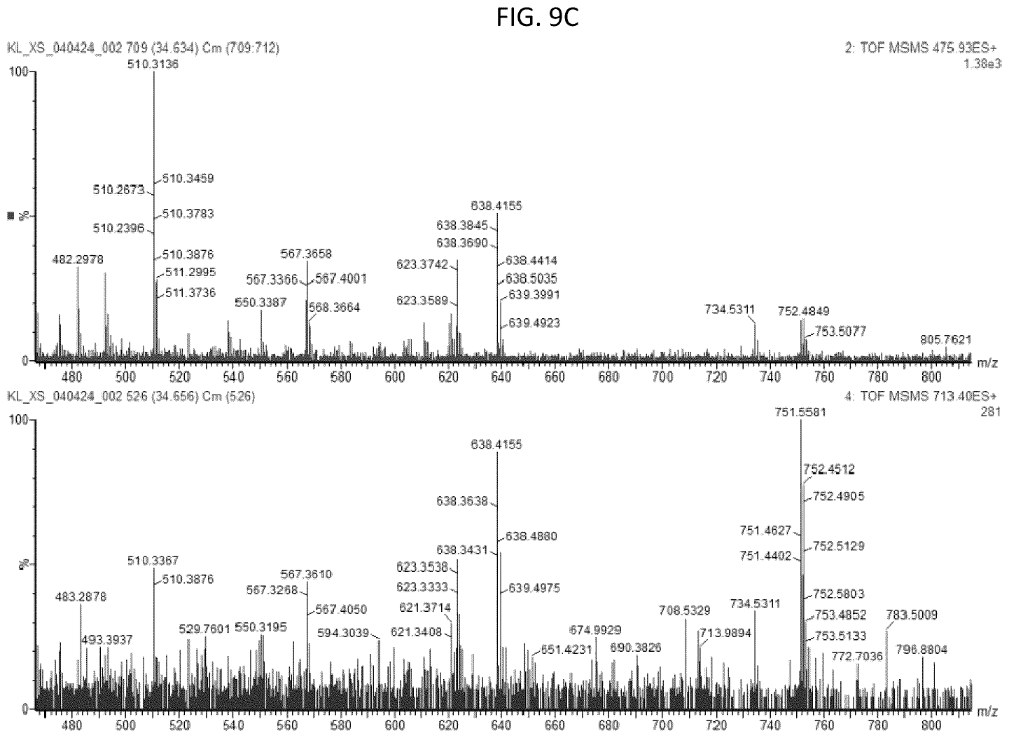

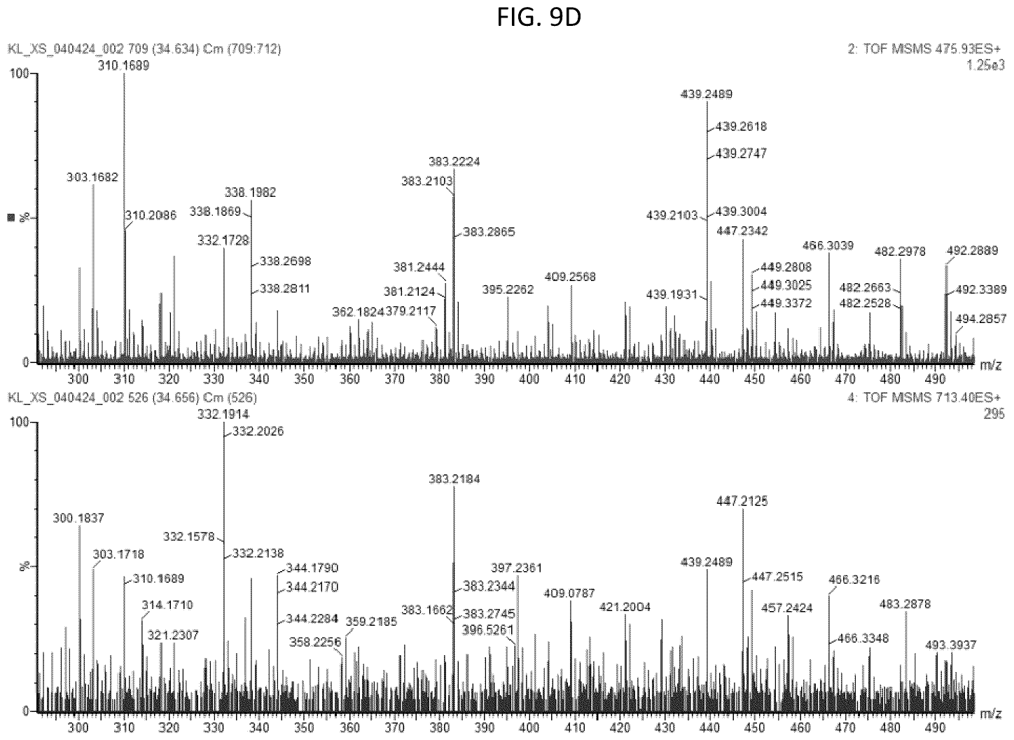

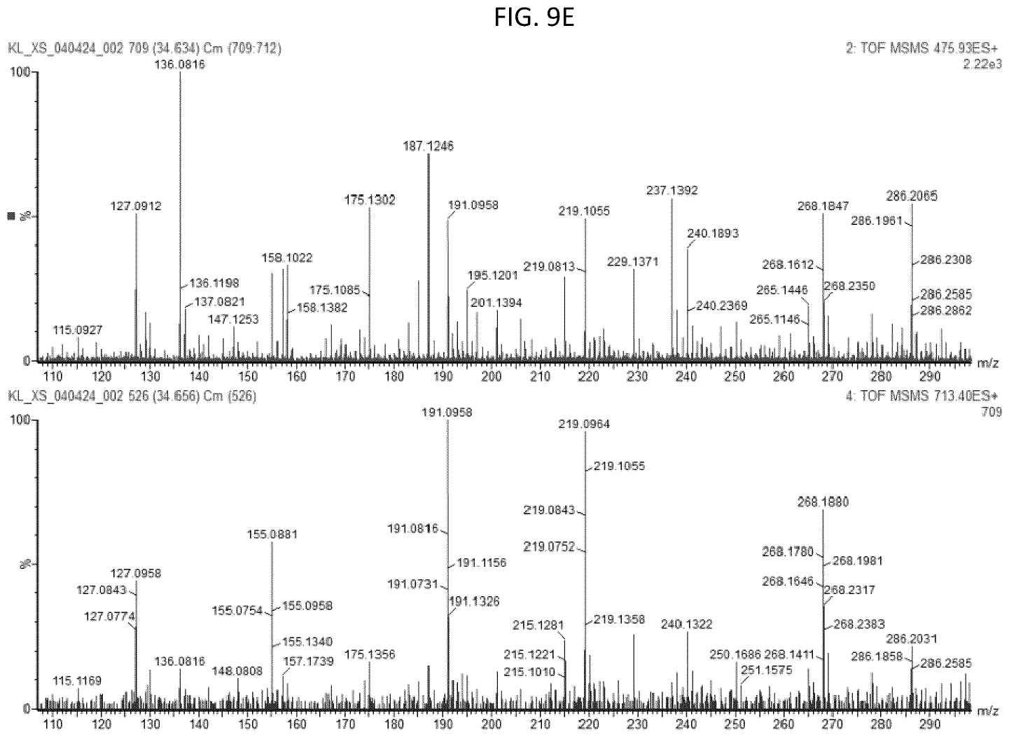

Further data is provided in FIGS. 7-9. Mascot database searches that support the identification of the desmin peptides and the novel desmin mutant are provided in FIG. 7. Expanded MS/MS spectra from FIG. 6 are shown in FIG. 8. Expanded MS/MS spectra of the precursor ions with m/z of 475.93 (3+) and 713.41 (2+) that correspond to peptide mALDVEIATYRK (SEQ ID NO: 2) and mALpYVEIATYR (SEQ ID NO: 4) are shown in FIG. 9.

### DISCUSSION

While considerable knowledge has been gained from genomic [18], transcriptomic [19] and proteogenomic [20] analysis of PDAC tumors, actionable biomarkers for this deadly cancer remain elusive [21]. For example, KRAS is mutated in 85% of PDAC [1]. But in a clinical trial where 38 PDAC patients with KRAS p.G12C-mutations were treated with sotorasib, a KRAS pG12C inhibitor, only 8 patients had an objective response. Median progression-free survival was 4.0 months, median overall survival 6.9 months, and 16 patients had adverse events [22].

Given PDAC's extensive genomic and transcriptomic heterogeneity [23], its lack of known TK drivers, and the multitude of TK approved drugs [9], we decided to screen for actionable TK protein biomarkers in human tumor biopsies using sensitive immunodetection in combination with MS. We were looking for RTKs activated by tyrosine phosphorylation [7], as well as protein substrates of Src which controls many cellular processes [24, 25].

We Found an Abundant Phosphoprotein, pTyr-Mutant Desmin D399Y in 2/6 PDAC Tumors

We used 1D/2D pTyr western blotting [8, 10], in combination with nanoLC-MS/MS [17], to screen for pTyr-protein biomarkers in six PDAC human tumor samples and five NAT controls purchased from a biobank. No high molecular weight pTyr-RTKs were enriched in PDAC tumors versus controls. However, this approach did detect a novel, abundant, 55 kDa pTyr-protein in 2/6 tumor samples and none of the NAT controls. The protein was identified by nanoLC-MS/MS as a pTyr-mutant desmin wherein aspartate 399 was replaced with a tyrosine, which was phosphorylated. Since orthogonal protein analysis methods (western blot and MS) were used to compliment and cross-confirm each other, this strengthens our conclusions.

Although serine/threonine phosphorylation of desmin by several kinases has been reported [26], and D399Y mutant desmin has been detected by a genome sequence analysis of a myopathy-affected family [27], to our knowledge this is the first report of pTyr399-mutant desmin in PDAC.

### This is a Perplexing Result

Desmin is a marker for activated pancreatic stellate cells (PSCs) [28], albeit a variable marker [3, 29], and not for PDAC tumor cells. PSCs, which are non-cancerous mesenchymal cells present in tumor stroma [30], do not have the array of mutated tumor suppressor genes (e.g., p53) that lead to the genomic instability of cancer cells [13]. Thus, while PSCs are known to express wild-type desmin [28], which was observed, they are unlikely to express mutated desmin. Yet pTyr-mutated desmin was an abundant protein in two PDAC tumor samples (FIG. 1D, FIGS. 2-4), which is provocative.

### Our Hypothesis

As reviewed by Hingorani [3], PSCs and PDAC tumor cells interact bidirectionally to generate desmoplasia. Mutant KRAS in tumor cells triggers the release of cytokines, chemokines, and growth factors that activate quiescent PSCs [31]. In turn the activated PSCs release extracellular vesicles (EVs) containing miR-21, which stimulate metastasis of the tumor cells [32-34].

We hypothesize that microRNA-21 stimulates both PDAC cancer cells and PSCs to express desmin, and that an unknown amount of the latter in cancer cells is mutant D399Y. Since we did not detect any unphosphorylated mutant desmin peptide, mALYVEIATYR (SEQ ID NO: 3), we further hypothesize that a SFK member immediately phosphorylated the mutant tyrosine. Although Src protein expression was only weakly correlated with pTyr-mutant desmin expression (FIG. 1C), SFKs may still be involved since regulation of their activity is complex [35], and since there are nine SFK members of which three (Src, Yes, and Fyn) are likely to be expressed [25]. Given that Yes Associated Protein (YAP1) expression is markedly increased in tumors 1 and 2 versus their controls (FIG. 1), the Yes kinase is a possible candidate.

The Clinical Proteomic Tumor Analysis Consortium (CPTAC) Did not Find pTyr-Mutant Desmin in their Proteogenomics Analysis of PDAC Tumors [20]

The words “desmin,” “phosphotyrosine,” and “pY” were not found in a text search of the CPTAC manuscript. pTyr-mutant desmin was either not detected by this group or was considered an unimportant passenger mutation. Thus, the abundant pTyr-mutant desmin that we found might be due to a passenger hotspot mutation [36] that has no effect on PDAC growth or metastasis.

Another possibility stems from differences in sample preparation. Since our system is compatible with SDS, our procedure (see Materials and Methods) includes homogenizing the tumor tissue in SDS buffer containing protease and phosphatase inhibitors along with nucleases. After homogenization, the tube is placed in a boiling water bath for five minutes to maximize protein solubilization. In many cases the homogenate clarifies. If not, the tubes are centrifuged to remove a small pellet of “cell debris”. Proteins in our homogenate were further resolved by 2D gel electrophoresis with WB and MS-compatible Coomassie staining to pinpoint the pTyr-proteins in the 2D pattern.

SDS is incompatible with MS instrumentation and must be either omitted or removed [37] from samples prior to MS analysis. Thus, the CPTAC group homogenized PDAC samples in 8M urea without heating followed by centrifugation to remove “cell debris”. It is possible the discarded centrifugation pellet contained undissolved membrane and cytoskeleton proteins.

What Advantage Might pTyr-Mutant Desmin Provide to Tumor Cells? Triggering Metastasis.

Desmin is a type II intermediate filament (IF). IFs are tissue-specific cytoskeleton components that reorganize in response to differentiation related signals [38]. Several IFs have been implicated in cancer epithelial mesenchymal transformation (EMT) and metastasis [39], but details of this complex process remain to be worked out [40]. Kraxner and Koster observed in a reconstituted system that serine/threonine phosphorylation of the intermediate filaments (IFs) desmin and vimentin causes disassembly and/or softening of the cell cytoskeleton, likely to promote an EMT and cell migration [41].

Allam et al. in reviewing pancreatic stellate cells in PDAC noted: “There is evidence that cancer cells promote their own non-malignant stroma to auto-facilitate their growth and spread: transformed malignant cells can acquire a mesenchymal-like phenotype expressing desmin, vimentin, and α-SMA and can camouflage like resident normal stromal cells [42].” [43].

Kraxner and Koster's observation provides a mechanism by which IF phosphorylation might promote metastasis. Allam et al.'s statement supports our hypothesis of pTyr-mutant desmin triggering metastasis. If the pTyr-mutant desmin present in PDAC cancer cells imparts a mesenchymal-like phenotype, these cells would be identified as STCs by IHC since desmin is a marker protein for these mesenchymal cells. However, since they are tumor cells expressing a phosphorylated IF protein (desmin) that promotes EMT/metastasis, then pTyr-mutant desmin abundance would increase as the metastatic cells multiply. This would explain the relatively high abundance of pTyr-desmin in our samples.

### Implications Applications: A Competitive Peptide Inhibitor Drug Might Reduce Metastasis

Given PDAC's extreme heterogeneity, the chain of events outlined here would likely only occur in a tumor subset. We observed pTyr-desmin in 2/6 samples in this report. However, if the hypothesis holds true that (1) SFKs phosphorylate all Y399 mutant desmin, and (2) pTyr-mutant desmin triggers metastasis, then a synthesized peptide drug (MALYVEIATYR (SEQ ID NO: 3) should interfere with Src phosphorylation of the mutant desmin protein and inhibit metastasis. Such a drug would likely have few side effects since it would interfere only with mutant desmin tyrosine phosphorylation and not with the function of wild-type desmin in muscle.

If the abundance of pTyr-mutant desmin observed here is due to metastatic overexpression of a subclone, then other similar desmin mutations would also be expanded and might serve as biomarkers that can be detected by IHC.

Limitations of this Study

While our identification of pTyr-mutant desmin in 2/6 tumor samples is unequivocal, the small size of this study is a limitation. The pTyr PTM could be present in a small percent of patients or could be an unimportant passenger mutation that has nothing to do with metastasis. While there is evidence in the literature that serine and threonine phosphorylation of intermediate filaments enhances metastasis [41], there is little data on tyrosine phosphorylation. As succinctly summarized by Bracken and Goodall, the EMT is a highly complex process key to tissue and organ development throughout the body. It is controlled by hundreds of transcription factors and microRNAs as well as by phosphorylation events.[40]. Unraveling the factors that trigger EMT in different cancers will not be easy.

### Future Directions

This preliminary study needs to be followed up and verified with a larger number of PDAC tumor samples and NAT controls using pTyr western blotting and MS. If possible, using research autopsy samples [44], where primary and metastatic tumor samples could be obtained from the same individual, would be useful. If pTyr-mutant desmin is detected in a subset of PDAC tumors and verified to trigger metastasis, then a specific antibody against the mutant desmin pTyr-peptide could be generated for IHC identification of this biomarker in patients.

### Conclusions and Impact

PDAC tumors are very heterogeneous, and their progression is based on stochastic tumor mutations that vary between individuals [23]. Stromal interactions are key as well [3]. Most cancer patients die because of metastatic events, and not due to primary tumors [45]. If we have stumbled onto a novel mechanism of PDAC metastasis, Src phosphorylation of mutated desmin, then a novel treatment becomes theoretically available. Competitive peptide inhibitors targeting the sequence mALYVEIATYR (SEQ ID NO: 3) would specifically interfere with SFK phosphorylation of mutant desmin and could be tailored to other desmin mutations if appropriate. Such peptide inhibitors could be synthesized and might have few side effects. Thus, if successful, this might provide a novel path to PDAC treatment.

### Materials and Methods:

### Sample Preparation

Five PDAC samples with matched normal adjacent tissue (NAT) were purchased from Spectrum Health Office of Research and Education, Grand Rapids, MI via Accio Biobank Online. A sixth PDAC sample P0, identically prepared, had been purchased in 2011 for another purpose without a NAT control from ILS Bio, LLC, now BioIVT) Chesterfield, MD and stored at −80° C. All tumor samples and controls were received on dry ice and stored at −80° C. until sample preparation described below.

Under the definition of human research subjects [45 CFR4 46.102(f)], the Office of Human Resource Protection does not consider research involving only coded private information or biospecimens (retrospective or remnant) to involve human subjects. These specimens were not specifically collected for the proposed research project. Because investigators cannot readily ascertain the identity of the individuals to whom the coded specimens pertain, the requirement for documented informed consent is not applicable.

Sample preparation was performed on ice with ice-cold reagents as follows: Tissue samples were rinsed with ice-cold Tris buffered saline containing 20 mM Tris, 500 mM NaCl, pH 7.5, then placed in a motorized glass-teflon homogenizer. The tissue was homogenized on ice with: Osmotic Lysis Buffer (10 mM Tris, 0.3% SDS) containing protease inhibitors (AEBSF, leupeptin, E-64, 5 mM EDTA, and benzamidine), Phosphatase Inhibitors I and II, RNase, DNase, and 5 mM MgCl₂) and then diluted 1:1 with SDS buffer (5% SDS, 10% glycerol, and 62.5 mM Tris, pH 6.8).

After homogenization and dilution, the sample tube was placed in a boiling water bath for 5 minutes, and the protein concentration was determined via the BCA assay (Pierce/Thermo Fisher). Finally, each sample was diluted to 4 mg/ml with SDS buffer containing 5% beta-mercaptoethanol. Aliquots were stored at −80° C.

### ID SDS PAGE

SDS slab gel electrophoresis and western blotting was carried out in 10% acrylamide slab gels (13×15 cm, 0.75 mm thick), as previously described for the second dimension of 2DE [8]. Electrophoresis was carried out for about 4 hours at 15 mA/gel. The following proteins (MilliporeSigma) were used as molecular weight standards in one lane on every gel: myosin (220,000), phosphorylase A (94,000), catalase (60,000), actin (43,000), carbonic anhydrase (29,000), and lysozyme (14,000). Western blot results were normalized by loading a constant amount of total protein, as recommended by NIH guidelines [46].

### 2D SDS PAGE

Two dimensional SDS PAGE was performed as previously described [8]. Briefly, isoelectric focusing was performed in polyacrylamide tube gels polymerized with 2% ampholines (130 mm long×2.3 mm internal diameter) sealed at the bottom with parafilm, were poured using 2% ampholines (Serva Electrophoresis, Heidelberg, Germany). Ampholines were pH 3-10 Iso-Dalt or a 1:1 mixture of pH 4-6 Ampholines and Servalyte pH 5-8. Samples were loaded at the top (basic end) of the polymerized acrylamide tube gel and isoelectric focusing carried out for 9600 volt-hrs.

Tube gels were extruded by air pressure and equilibrated for 10 minutes in buffer “O” (10% glycerol, 50 mM dithiothreitol, 2.3% SDS and 0.0625 M tris, pH 6.8). The equilibrated tube gels were frozen on dry ice to prevent protein diffusion and thawed immediately before loading onto the 2^{nd} dimension slab gel. Each tube gel was sealed in 1 ml of agarose to the top of a stacking gel overlaying a 10% acrylamide slab gel and electrophoresis carried out for about 4 hours at 15 mA/gel. Molecular weight standards were loaded in an agarose well on the basic end of the tube gel. One μg of an IEF internal standard, tropomyosin, was loaded with every sample.

### Western Blotting

After slab gel electrophoresis, gels were placed in transfer buffer (10 mM CAPS, pH 11.0, 10% methanol) and transblotted onto PVDF membranes overnight at 200 mA and approximately 100 volts/two gels. The blots were stained with Coomassie Brilliant Blue R-250 (Sigma-Aldrich) and scanned.

Coomassie blue stained PVDF membranes were wet in 100% methanol to remove the stain, rinsed in tween-20 (Biorad) TBS (TTBS), and blocked for two hours in 5% non-fat dry milk diluted in TTBS. Primary antibody incubations were performed overnight on an orbital shaker in 2% nonfat dry milk using antibody dilutions shown in the figures. Blots were rinsed 3×10 minutes in TTBS and incubated with secondary antibody diluted 1:2000 for 2 hours. Finally, the blots were treated with Pierce ECL reagent (ThermoFisher) and exposed to x-ray film [Kodak BioMax XAR film (ThermoFisher) or GE Amersham Hyperfilm ECL], followed by film development with an automatic Konica Minolta Medical Film Processor SRX-101A.

### Mass Spectrometry

Gel spots were digested with trypsin and the resulting peptides mixtures were analyzed by a NanoAcquity UPLC coupled with a QTOF Xevo G2-XS mass spectrometer (both from Waters Corp) according to published procedures [8]. The raw data were converted into mzML files using MS convert and the database search was done against NCBI Human database using propionamide as fixed modification and methionine oxidation and phosphoY/S/T as variable modifications.

### REFERENCES

1. Luo, J., KRAS mutation in pancreatic cancer. Semin Oncol, 2021. 48(1): p. 10-18.
2. Joshi, V. B., O. L. Gutierrez Ruiz, and G. L. Razidlo, The Cell Biology of Metastatic Invasion in Pancreatic Cancer: Updates and Mechanistic Insights. Cancers (Basel), 2023. 15(7).
3. Hingorani, S. R., Epithelial and stromal co-evolution and complicity in pancreatic cancer. Nat Rev Cancer, 2023. 23(2): p. 57-77.
4. Mao, W., et al., YAP in pancreatic cancer: oncogenic role and therapeutic strategy. Theranostics, 2021. 11(4): p. 1753-1762.
5. Rozengurt, E. and G. Eibl, Crosstalk between KRAS, SRC and YAP Signaling in Pancreatic Cancer: Interactions Leading to Aggressive Disease and Drug Resistance. Cancers (Basel), 2021. 13(20).
6. Ebelt, N. D., V. Zamloot, and E. R. Manuel, Targeting desmoplasia in pancreatic cancer as an essential first step to effective therapy. Oncotarget, 2020. 11(38): p. 3486-3488.
7. Kendrick, N., et al., Detection of Activated Receptor Tyrosine Kinases in Human Lung Squamous Cell Carcinoma. bioRxiv, 2023: p. 2023.02.07.527536.
8. Kendrick, N., et al., Preparation of a phosphotyrosine-protein standard for use in semiquantitative western blotting with enhanced chemiluminescence. PLoS One, 2020. 15(6): p. e0234645.
9. Huang, L., S. Jiang, and Y. Shi, Tyrosine kinase inhibitors for solid tumors in the past 20years (2001-2020). J Hematol Oncol, 2020. 13(1): p. 143.
10. Kendrick, N., et al., 2D SDS PAGE in Combination with Western Blotting and Mass Spectrometry Is a Robust Method for Protein Analysis with Many Applications. Adv Exp Med Biol, 2019. 1140: p. 563-574.
11. Zhou, Y. and J. F. Hancock, RAS nanoclusters are cell surface transducers that convert extracellular stimuli to intracellular signalling. FEBS Lett, 2023. 597(6): p. 892-908.
12. Taddei, M. L., et al., Role of tyrosine phosphorylation in modulating cancer cell metabolism. Biochim Biophys Acta Rev Cancer, 2020. 1874(2): p. 188442.
13. Weinberg, R. A., et al., The biology of cancer. Third edition, International student edition. ed. 2023, New York, N.Y: W. W. Norton & Company. xxi, 865, 8, 48, 40 pages: illustrations (chiefly colour).
14. Kleinert, P., et al., Effect of glycosylation on the protein pattern in 2-D-gel electrophoresis. Proteomics, 2007. 7(1): p. 15-22.
15. Deng, X., et al., The challenge to quantify proteins with charge trains due to isoforms or conformers. Electrophoresis, 2012. 33(2): p. 263-9.
16. Li, J. and X. Zhan, Mass spectrometry analysis of phosphotyrosine-containing proteins. Mass Spectrom Rev, 2023.
17. Lux, J. C., et al., Identification of dysregulation of atrial proteins in rats with chronic obstructive apnea using two-dimensional polyacrylamide gel electrophoresis and mass spectrometry. J Cell Mol Med, 2019. 23(4): p. 3016-3020.
18. Dreyer, S. B., et al., Pancreatic Cancer Genomes: Implications for Clinical Management and Therapeutic Development. Clin Cancer Res, 2017. 23(7): p. 1638-1646.
19. Bailey, P., et al., Genomic analyses identify molecular subtypes of pancreatic cancer. Nature, 2016. 531(7592): p. 47-52.
20. Cao, L., et al., Proteogenomic characterization of pancreatic ductal adenocarcinoma. Cell, 2021. 184(19): p. 5031-5052 e26.
21. Gyawali, B. and C. M. Booth, Treatment of metastatic pancreatic cancer: 25 years of innovation with little progress for patients. Lancet Oncol, 2024. 25(2): p. 167-170.
22. Strickler, J. H., et al., Sotorasib in KRAS p.G12C-Mutated Advanced Pancreatic Cancer. N Engl J Med, 2023. 388(1): p. 33-43.
23. Connor, A. A. and S. Gallinger, Pancreatic cancer evolution and heterogeneity: integrating omics and clinical data. Nat Rev Cancer, 2022. 22(3): p. 131-142.
24. Pelaz, S. G. and A. Tabernero, Src: coordinating metabolism in cancer. Oncogene, 2022. 41(45): p. 4917-4928.
25. Poh, A. R. and M. Ernst, Functional roles of SRC signaling in pancreatic cancer: Recent insights provide novel therapeutic opportunities. Oncogene, 2023. 42(22): p. 1786-1801.
26. Winter, D. L., et al., Posttranslational modifications of desmin and their implication in biological processes and pathologies. Histochem Cell Biol, 2014. 141(1): p. 1-16.
27. Goudeau, B., et al., Variable pathogenic potentials of mutations located in the desmin alpha-helical domain. Hum Mutat, 2006. 27(9): p. 906-13.
28. Haeberle, L., et al., Stromal heterogeneity in pancreatic cancer and chronic pancreatitis. Pancreatology, 2018. 18(5): p. 536-549.
29. Thomas, D. and P. Radhakrishnan, Pancreatic Stellate Cells: The Key Orchestrator of The Pancreatic Tumor Microenvironment. Adv Exp Med Biol, 2020. 1234: p. 57-70.
30. Pothula, S. P., et al., Pancreatic stellate cells: Aiding and abetting pancreatic cancer progression. Pancreatology, 2020. 20(3): p. 409-418.
31. Tape, C. J., et al., Oncogenic KRAS Regulates Tumor Cell Signaling via Stromal Reciprocation. Cell, 2016. 165(7): p. 1818.
32. Sarkar, R., et al., Emerging role of pancreatic stellate cell-derived extracellular vesicles in pancreatic cancer. Semin Cancer Biol, 2023. 93: p. 114-122.
33. Ali, A., et al., Prognostic implications of microRNA-21 overexpression in pancreatic ductal adenocarcinoma: an international multicenter study of 686 patients. Am J Cancer Res, 2022. 12(12): p. 5668-5683.
34. Ma, Q., et al., Upregulation of exosomal microRNA-21 in pancreatic stellate cells promotes pancreatic cancer cell migration and enhances Ras ERK pathway activity. Int J Oncol, 2020. 56(4): p. 1025-1033.
35. Okada, M., Regulation of the SRC family kinases by Csk. International journal of biological sciences, 2012. 8(10): p. 1385-97.
36. Hess, J. M., et al., Passenger Hotspot Mutations in Cancer. Cancer Cell, 2019. 36(3): p. 288-301 e14.
37. Wisniewski, J. R., et al., Universal sample preparation method for proteome analysis. Nature methods, 2009. 6(5): p. 359-62.
38. Dutour-Provenzano, G. and S. Etienne-Manneville, Intermediate filaments. Curr Biol, 2021. 31(10): p. R522-R529.
39. Sharma, P., et al., Intermediate Filaments as Effectors of Cancer Development and Metastasis: A Focus on Keratins, Vimentin, and Nestin. Cells, 2019. 8(5).
40. Bracken, C. P. and G. J. Goodall, The many regulators of epithelial-mesenchymal transition. Nat Rev Mol Cell Biol, 2022. 23(2): p. 89-90.
41. Kraxner, J. and S. Koster, Influence of phosphorylation on intermediate filaments. Biol Chem, 2023.
42. Petersen, O. W., et al., Epithelial to mesenchymal transition in human breast cancer can provide a nonmalignant stroma. Am J Pathol, 2003. 162(2): p. 391-402.
43. Allam, A., et al., Pancreatic stellate cells in pancreatic cancer: In focus. Pancreatology, 2017. 17(4): p. 514-522.
44. Duregon, E., et al., Rapid research autopsy is a stealthy but growing contributor to cancer research. Cancer, 2019. 125(17): p. 2915-2919.
45. Gerstberger, S., Q. Jiang, and K. Ganesh, Metastasis. Cell, 2023. 186(8): p. 1564-1579.
46. Fosang, A. J. and R. J. Colbran, Transparency Is the Key to Quality. J Biol Chem, 2015. 290(50): p. 29692-4.

What is claimed:

1. A method for detecting a mutant desmin protein comprising a phosphorylated D399Y mutation, the method comprising:
obtaining a sample from a subject; and
detecting the mutant desmin protein in the sample, wherein the mutant desmin protein comprises a tyrosine residue at position 399 that is phosphorylated.

2. The method of claim 1, wherein the sample is a pancreatic tissue sample.

3. The method of claim 1, wherein detecting the mutant desmin protein comprises using mass spectrometry.

4. The method of claim 1, wherein detecting the mutant desmin protein comprises using an antibody-based detection method.

5. The method of claim 4, wherein the antibody-based detection method is selected from the group consisting of western blotting, immunohistochemistry, and enzyme-linked immunosorbent assay.

6. The method of claim 1, further comprising a step of homogenizing the sample in a buffer comprising sodium dodecyl sulfate prior to detecting the mutant desmin protein and optionally further comprising a step of boiling the homogenized sample prior to the detecting step.

7. The method of claim 1, wherein the detection of the mutant desmin protein comprising a tyrosine residue at position 399 is indicative of a myopathy, optionally a cardiomyopathy.

8. The method of claim 2, wherein the method is used to diagnose pancreatic ductal adenocarcinoma in the subject.

9. A composition comprising a fragment of a mutant desmin protein, wherein the fragment comprises a phosphorylated tyrosine residue at position 399 corresponding to a D399Y mutation in wild-type desmin.

10. The composition of claim 9, wherein the fragment comprises the amino acid sequence MALYVEIATYR (SEQ ID NO: 3).

11. The composition of claim 9, wherein the fragment is between 5 and 20 amino acids in length.

12. The composition of claim 9, further comprising a pharmaceutically acceptable carrier.

13. The composition of claim 12, wherein the pharmaceutically acceptable carrier is selected from the group consisting of liposomes, nanoparticles, and extracellular vesicles.

14. The composition of claim 13, wherein the composition is formulated for therapeutic use in treating cancer and the cancer is optionally a pancreatic ductal adenocarcinoma.

15. A method of treating cancer in a subject, the method comprising:
administering to the subject the composition of claim 9.

16. The method of claim 15, wherein the cancer is pancreatic ductal adenocarcinoma.

17. The method of claim 16, wherein the fragment comprises the amino acid sequence MALYVEIATYR.

18. The method of claim 17, wherein the composition is administered intravenously.

19. The method of claim 15, wherein the subject has a cancer expressing a mutant desmin protein comprising a phosphorylated D399Y mutation.

20. A construct comprising a polynucleotide encoding at least a portion of SEQ ID NO: 6 operably connected to a promoter, wherein the portion of SEQ ID NO: 6 comprises at least 8 amino acids and includes amino acid 399Y.


SEQUENCE LISTING (6 sequences)

SEQ ID NO: 1 - protein, 470 aa, Homo sapiens
MSQAYSSSQRVSSYRRTFGGAPGFPLGSPLSSPVFPRAGFGSKGSSSSVTSRVYQVSRTSGGAGGLGSLRASRLGTTRTPSSYGAGELLDFSLADAVNQEFLTTRTNEKVELQELNDRFANYIEKVRFLEQQNAALAAEVNRLKGREPTRVAELYEEELRELRRQVEVLTNQRARVDVERDNLLDDLQRLKAKLQEEIQLKEEAENNLAAFRADVDAATLARIDLERRIESLNEEIAFLKKVHEEEIRELQAQLQEQQVQVEMDMSKPDLTAALRDIRAQYETIAAKNISEAEEWYKSKVSDLTQAANKNNDALRQAKQEMMEYRHQIQSYTCEIDALKGTNDSLMRQMRELEDRFASEASGYQDNIARLEEEIRHLKDEMARHLREYQDLLNVKMALDVEIATYRKLLEGEESRINLPIQTYSALNFRETSPEQRGSEVHTKKTVMIKTIETRDGEVVSEATQQQHEVL

SEQ ID NO: 2 - protein, 12 aa, Homo sapiens
MALDVEIATYRK

SEQ ID NO: 3 - protein, 11 aa, Homo sapiens
MALYVEIATYR

SEQ ID NO: 4 - protein, 11 aa, Homo sapiens
  MOD_RES 4: note = PHOSPHORYLATION
MALYVEIATYR

SEQ ID NO: 5 - protein, 12 aa, Homo sapiens
  MOD_RES 4: note = PHOSPHORYLATION
MALYVEIATYRK

SEQ ID NO: 6 - protein, 470 aa, Homo sapiens [sequence omitted]